(19) United States
(12) Patent Application Publication
(10) Pub. No.: US 2015/0018237 A1
(43) Pub. Date: Jan. 15, 2015

(54) MEANS AND METHODS FOR ASSESSING BONE DISORDERS

(71) Applicant: BASF SE, Ludwigshafen (DE)

(72) Inventors: Tilmann B. Walk, Kleinmachnow (DE); Bennard van Ravenzwaay, Altrip (DE); Werner Mellert, Hassloch (DE); Eric Fabian, Speyer (DE); Volker Strauss, Bad Durkheim (DE); Hennicke Kamp, Bischheim (DE); Jan C. Wiemer, Berlin (DE); Ralf Looser, Berlin (DE); Michael Manfred Herold, Berlin (DE); Alexandre Prokoudine, Berlin (DE)

(73) Assignee: BASF SE, Ludwigshafen (DE)

(21) Appl. No.: 14/377,016

(22) PCT Filed: Feb. 15, 2013
(86) PCT No.: PCT/IB2013/051228

§ 371 Date: Aug. 6, 2014

Related U.S. Application Data
(60) Provisional application No. 61/598,960, filed on Feb. 15, 2012.

(30) Foreign Application Priority Data
Feb. 15, 2012 (EP) 12155631.0

(51) Int. Cl. G01N 33/68; G01N 33/92; G01N 33/50; G01N 33/84

(52) U.S. Cl. CPC G01N 33/6893; G01N 33/84; G01N 33/92; G01N 33/6806; G01N 33/5044; G01N 2570/00; G01N 2800/108
USPC 506/9; 436/501; 435/7.92; 435/7.94; 506/12; 435/287.2; 422/69; 422/82.01; 506/18

(57) ABSTRACT

The present invention pertains to the field of diagnostics for bone disorders and toxicological assessments for risk stratification of chemical compounds. Specifically, it relates to a method for diagnosing a bone disorder. It also relates to a method for determining whether a compound is capable of inducing such a bone disorder in a subject and to a method of identifying a drug for treating a bone disorder. Furthermore, the present invention relates to a device and a kit for diagnosing a bone disorder.

The present invention pertains to the field of diagnostics for bone disorders and toxicological assessments for risk stratification of chemical compounds. Specifically, it relates to a method for diagnosing a bone disorder. It also relates to a method for determining whether a compound is capable of inducing such a bone disorder in a subject and to a method of identifying a drug for treating a bone disorder. Furthermore, the present invention relates to a device and a kit for diagnosing a bone disorder.

Bone consists of 35% organic matrix and 65% minerals, predominantly hydroxyapatite. The organic matrix is made up to 90% of type 1 collagen and up to 10% of non-collagenous proteins. About half of the non-collagenous proteins comprises osteocalcin (bone Gla protein), osteonectin and osteopontin as well as bone sialo-protein with equal shares (Marcus et al, 1996).

In adults 5-10% of the skeleton is remodeled per year, kept in balance by bone formation established by osteoblasts and bone resorption which is performed by osteoclasts. Osteoblasts derive from mesenchymal stem cells. They are synthesizing the non-collagen as well as the type 1 collagen proteins and they manage the calcification of the organic matrix. The multi-nucleated osteoclasts originate from hemapoietic stem cells. They initiate the demineralization of bone tissue by excretion of hydrogen ions followed by degradation of the exposed organic bone matrix by proteolytic enzymes of this cell type (Bilezikian et al, 1996).

Osteoporosis (i.e., loss of bone mass) affects more than 75 million people in Europe, Japan and USA. It is three times more common in women than in men. A substantial bone loss is usually beginning in 65 years old men and 50 year old women (Rizzoli and Bonjour, 1999). In Europe the number of women older than 50 years is projected to increase by 30-40% between 1990 and 2025. This trend is even more marked in other areas of the world. In 1990, an estimated 1.3-1.7 million hip fractures occurred worldwide, mainly as a consequence of osteoporosis. This number is expected to increase at least to 3 million in 2025 (Cooper et al., 1992; Gullberg et al., 1997).

Bone metabolism disorders can be divided according to its cause in (i) primary mineralization deficiencies and (ii) loss of organic bone matrix:

(i) Skeleton mineralization is diminished by nutritional deficiencies of calcium or phosphate as well as decreased synthesis of calcitriol due to a lack of vitamin D because of reduced exposure of the skin to sun light. The disease is called rickets (rachitis) in children and osteomalacia in adults. Demineralization of the bones can be caused also by increased activity of the parathyroid hormone (PTH). The hyperparathyroidism (HPT) may be due to neoplasms in the parathyroidal glands (primary HPT) or as feedback to decreased intestinal absorption or increased renal loss of calcium (secondary HPT). A long-lasting secondary HPT may result in a hyperplasia of the parathyroid tissue with autonomous PTH secretion (tertiary HPT).

(ii) The main causes of systemic osteoporosis are estrogen or androgen deficiencies in menopausal women and men (Greendale et la., 1997; Slemenda et al., 1997). Other endocrine reasons for osteoporosis may be increased thyroid hormone activities (Britto et al., 1994) and high concentrations of iatrogen administered or endogen glucocorticoids (Cushing's Syndrome) (Adinoff and Hollister, 1983; Canalis, 1996). Low protein intake as well as high protein loss because of renal insufficiency are also factors aggravating osteoporosis (Bonjour et al., 1997) as well as administration of some drugs (cholestyramine (lipid-lowering drug; Compston and Horton, 1978), cyclosporine (immunosuppressant drug; Shane et al., 1993), phenobarbitone and carbamazepin (antiepileptic drugs; Weinstein et al., 1984), etc.) as well as high consumption of ethanol and cigarettes.

Diagnosis of bone metabolism disorders in humans can be established by measuring bone mineral density with clinical methods like conventional radiography as well as single or dual energy X-ray absorptiometry (SXA, DXA or DEXA) as well as quantitative computed tomography (QCT) and quantitative ultrasound (QUS) (WHO, 2003).

Measuring clinical pathology parameters in blood and urine may help to find the reason for bone mass loss. They can also be applied as a diagnostic screening tool and for the monitoring of an anti-osteoporotic therapy (Delmas et al., 2000).

The clinical pathology parameters can be divided in (i) bone formation and (ii) bone resorption markers as well as (iii) mineralization parameters.

(i) Bone formation markers are bone specific alkaline phosphatase, osteocalcin (=bone gla protein; intact or N-mid fragment), type I collagen propeptides (PINP and PICP).

(ii) Bone resorption parameters are urinary hydroxyproline, pyridinoline and deoxypyridinoline, hydroxylysin (and its glycosids), type I collagen telopeptides (NTX, CTX, ICTP), bone sialoprotein, tartrate-resistant acid phosphatase type 5b (Bone TRAP).

(iii) Bone mineralization markers are urinary calcium and phosphate, parathyroid hormone (PTH), calcitonin, 25 Hydroxy-vitamin D, 1,25 Dihydroxy-vitamin D.

Some but not all mentioned parameters can be measured in humans as well as in rodents.

Presently, a panel of clinical pathology parameters mentioned above have to be measured in subsequent test runs in serum/plasma and urine at several time points in order to estimate the influence of a compound on bone metabolism. A simple and sensitive technique is necessary to give answers on the influence on bone metabolism with a single routine measurement (see Adinoff, A. D., Hollister, J. R. (1983) Steroid-induced fractures and bone loss in patients with asthma. N Engl J Med, 309, 265-268; Bilezikian, J. P., Raisz, L. G., Rodan, G. A., (eds.) Principles of bone biology, San Diego, Calif., Academic Press, 1996; Bonjour, J. P., Schuerch, M. A., Chevalley, T., Ammann, P., Rizzoli, R. (1997) Protein intake, IGF-1 and osteoporosis. Osteoporosis International, 1997, 7 (suppl 3), S36-S42; Britto, J. M. Fenton, A. J., Holloway, W. R., Nicholson G. C. (1994) Osteoblasts mediate thyroid hormone stimulation of osteoclastic bone resorption. Endocrinology, 134, 169-176; Canalis, E. (1996) Mechanisms of glucocorticoid action in bone: implications to glucocorticoid-induced osteoporosis. Clin Endocrinol Metab, 81, 3441-3447; Compston, J. E., Horton, L. W. (1978) Oral 25-hydroxyvitmain D3 in treatment of osteomalacia associated with ileal resction and cholestyramine therapy. Gastroenterology, 74, 900-902; Cooper, C., Campion, G., Melton L. J. (1992) Hip fractures in the elderly: a worldwide projection. Osteoporosis International, 2, 285-289; Delmas, P. D., Eastell, R., Garnero, P., Seibel, M. J., Stepan, J. (2000), The use of biochemical markers of bone turnover in osteoporosis. Osteoporosis International, Suppl 6, S2-517; Greendale, G. A., Edelstein, S., Barrett-Connor, E. (1997) Endogenous sex steroids and bone mineral density in older women and men: the Rancho Bernardo study, Journal of Bone & Mineral Research, 12, 1833-1843; Gullberg, B., Johnell, O., Kanis, J. A. (1997) Worldwide projections for hip fracture. Osteoporosis International, 7, 407-413; Marcus, R., Feldman, D., Kelsey, J. (eds.) Osteoporosis, San Diego, Calif., Academic Press, 1996; Rizzoli, R., Bonjour, J. P. (1999) Determinants of peak bone mass and mechanisms of bone loss. Osteoporosis International, 9 (suppl. 2), S17-S23; Shane, E., Rivas, M. D. C., Silverberg, S. J., Kim, T. S., Staron, R. B., Bilezikian, J. P. (1993) Osteoporosis after cardiac transplantation. Am J Med, 94 (3), 257-264; Slemenda, C. W., Longcope, C., Zhou, L., Peacock, M., Johnston, C. C. (1997) Sex steroids and bone mass in older men. Positive associations with serum estrogens and negative associations with androgens. J Clin Invest, 100 (7), 1755-1759; Weinstein, R. S., Bryce, G. F., Sappington, L. J., King, D. W., Gallagher, B. B. (1984) Decreased serum ionized calcium and normal vitamin D metabolite levels with anticonvulsant drug treatment. J Clin Endocrinol Metab, 58 (6), 1003-1009; WHO Technical Report Series No. 921 Prevention and Management of Osteoporosis, Geneva, 2003).

Sensitive and specific methods for determining efficiently and reliably bone disorders and, in particular, the early onset thereof are not available but would, nevertheless, be highly appreciated. The importance of bone disorders may become apparent if one considers its consequences on hematopoiesis including lymphopoiesis. Moreover, chemical compounds which are used in any kind of industry in the European Community, e.g., will now need to comply with REACH (Registration, Evaluation and Authorisation of Chemicals). In other countries, similar toxicological risk assessments need to be done, e.g., the Material Safety Data Sheets (MSDS) in the US. It will be understood that the potential of a chemical compound to induce bone disorders with regards to mineralization will be deemed as a high risk for the compound and, consequently, the compound will be available only for limited applications and when obeying high security standards.

Sensitive and specific methods for assessing the toxicological properties of a chemical compound and, in particular, bone disorder, in an efficient and reliable manner are not yet available but would, nevertheless, be highly appreciated.

Thus, the technical problem underlying the present invention could be seen as the provision of means and methods for complying with the aforementioned needs. The technical problem is solved by the embodiments characterized in the claims and described herein below.

Accordingly, the present invention relates to a method for diagnosing a bone disorder comprising:

(a) determining the amount of at least one biomarker selected from any one of Tables 1a, 1b, 1c, 1d, 2a, 2b, 2c, or 2d in a test sample of a subject suspected to suffer from a bone disorder, and (b) comparing the amounts determined in step (a) to a reference, whereby the bone disorder is to be diagnosed.

In a particular embodiment of the method of the invention, a method is provided for diagnosing a bone disorder comprising:

(a) selecting a male or female subject suspected to suffer from a bone disorder;

(b) obtaining a test sample from said selected subject;

(c) pre-treating said sample in preparation for analysis;

(d) determining the amount of at least one biomarker selected from any one of Tables 1a, 1b, 1c, 1d, 2a, 2b, 2c, or 2d in said test sample, and

(e) comparing the amounts determined in step (d) to a reference; and

(f) based on the comparison of step (e), diagnose a bone disorder by monitoring, confirmation or classification of the a bone disorder or its symptoms.

In a preferred embodiment of the aforementioned method said subject has been brought into contact with a compound suspected to be capable of inducing bone disorder.

The present invention also relates to a method of determining whether a compound is capable of inducing bone disorder in a subject comprising:

(a) determining in a sample of a subject which has been brought into contact with a compound suspected to be capable of inducing bone disorder the amount of at least one biomarker selected from any one of Tables 1a, 1b, 1c, 1d, 2a, 2b, 2c, or 2d; and

(b) comparing the amounts determined in step (a) to a reference, whereby the capability of the compound to induce bone disorder is determined.

In a particular embodiment of the method of the invention, a method is provided for determining whether a compound is capable of inducing a bone disorder in a subject comprising:

(a1) (i) selecting a male or female subject;

(ii) bringing said subject into contact with a compound suspected to be capable of inducing a bone disorder, or

(a2) selecting a male or female subject brought into contact with a compound capable of inducing a bone disorder;

(b) obtaining a test sample from said selected subject;

(c) pre-treating said sample in preparation for analysis;

(d) determining the amount of at least one biomarker selected from any one of Tables 1a, 1b, 1c, 1d, 2a, 2b, 2c, or 2d in said test sample, and

(e) comparing the amounts determined in step (d) to a reference; and

(f) based on the comparison of step (e), identifying whether the compound is capable of inducing a bone disorder, or not.

In a preferred embodiment of the aforementioned method said compound is at least one compound selected from the group consisting of: Sodium perchlorate, Sodium fluoride, Calcitonin (Salmon) and Calcium carbonate.

In another preferred embodiment of the methods of the present invention said reference is derived from (i) a subject or group of subjects which suffers from bone disorder or (ii) a subject or group of subjects which has been brought into contact with at least one compound selected from the group consisting of: Sodium perchlorate, Sodium fluoride, Calcitonin (Salmon) and Calcium carbonate. In a more preferred embodiment of said method essentially identical amounts for the biomarkers in the test sample and the reference are indicative for bone disorder.

In another preferred embodiment of the methods of the present invention said reference is derived from (i) a subject or group of subjects known to not suffer from bone disorder or (ii) a subject or group of subjects which has not been brought into contact with at least one compound selected from the group consisting of: Sodium perchlorate, Sodium fluoride, Calcitonin (Salmon) and Calcium carbonate. In a more preferred embodiment of said methods amounts for the biomarkers which differ in the test sample in comparison to the reference are indicative for bone disorder.

In yet another embodiment of the methods of the present invention said reference is a calculated reference for the biomarkers for a population of subjects. In a more preferred embodiment of said methods amounts for the biomarkers which differ in the test sample in comparison to the reference are indicative for bone disorder.

The present invention also contemplates a method of identifying a substance for treating bone disorder comprising the steps of:

(a) determining in a sample of a subject suffering from bone disorder which has been brought into contact with a candidate substance suspected to be capable of treating bone disorder the amount of at least one biomarker selected from any one of Tables 1a, 1b, 1c, 1d, 2a, 2b, 2c, or 2d; and

(b) comparing the amounts determined in step (a) to a reference, whereby a substance capable of treating bone disorder is to be identified.

In a particular embodiment of the method of the invention, a method is provided for identifying a substance for treating a bone disorder comprising:

(a1) (i) selecting a male or female subject;

(ii) bringing said subject into contact with a compound suspected to be capable of inducing a bone disorder such that a bone disorder is elicited, or

(a2) selecting a male or female suffering from a bone disorder;

(b) obtaining a test sample from said selected subject;

(c) pre-treating said sample in preparation for analysis;

(d) determining the amount of at least one biomarker selected from any one of Tables 1a, 1b, 1c, 1d, 2a, 2b, 2c, or 2d in said test sample, and

(e) comparing the amounts determined in step (d) to a reference; and

(f) based on the comparison of step (e), identifying and selecting the substance for treating a bone disorder.

In a preferred embodiment of the aforementioned method said reference is derived from (i) a subject or group of subjects which suffers from bone disorder or (ii) a subject or group of subjects which has been brought into contact with at least one compound selected from the group consisting of: Sodium perchlorate, Sodium fluoride, Calcitonin (Salmon) and Calcium carbonate. In a more preferred embodiment of said method amounts for the biomarkers which differ in the test sample and the reference are indicative for a substance capable of treating bone disorder.

In another preferred embodiment of the aforementioned method said reference is derived from (i) a subject or group of subjects known to not suffer from bone disorder or (ii) a subject or group of subjects which has not been brought into contact with at least one compound selected from the group consisting of: Sodium perchlorate, Sodium fluoride, Calcitonin (Salmon) and Calcium carbonate. In a more preferred embodiment of the said methods essentially identical amounts for the biomarkers in the test sample and the reference are indicative for a substance capable of treating bone disorder.

In yet another preferred embodiment of the aforementioned method said reference is a calculated reference for the biomarkers in a population of subjects. In a more preferred embodiment of the said methods essentially identical amounts for the biomarkers in the test sample and the reference are indicative for a substance capable of treating bone disorder.

The present invention also relates to the use of at least one biomarker selected from any one of Tables 1a, 1b, 1c, 1d, 2a, 2b, 2c, or 2d or a detection agent for the said biomarker for diagnosing bone disorder in a sample of a subject.

Moreover, the present invention relates to a device for diagnosing bone disorder in a sample of a subject suspected to suffer therefrom comprising:

(a) an analyzing unit comprising a detection agent for at least one biomarker selected from any one of Tables 1a, 1b, 1c, 1d, 2a, 2b, 2c, or 2d which allows for determining the amount of the said biomarker present in the sample; and, operatively linked thereto,

(b) an evaluation unit comprising a stored reference and a data processor which allows for comparing the amount of the said at least one biomarker determined by the analyzing unit to the stored reference, whereby bone disorder is diagnosed.

In a preferred embodiment of the device of the invention said stored reference is a reference derived from a subject or a group of subjects known to suffer from bone disorder or a subject or group of subjects which has been brought into contact with at least one compound selected from the group consisting of Sodium perchlorate, Sodium fluoride, Calcitonin (Salmon) and Calcium carbonate, and said data processor executes instructions for comparing the amount of the at least one biomarker determined by the analyzing unit to the stored reference, wherein an essentially identical amount of the at least one biomarker in the test sample in comparison to the reference is indicative for the presence of bone disorder or wherein an amount of the at least one biomarker in the test sample which differs in comparison to the reference is indicative for the absence of bone disorder.

In another preferred embodiment of the device of the invention said stored reference is a reference derived from a subject or a group of subjects known to not suffer from bone disorder or a subject or group of subjects which has not been brought into contact with at least one compound selected from the group consisting of Sodium perchlorate, Sodium fluoride, Calcitonin (Salmon) and Calcium carbonate, and said data processor executes instructions for comparing the amount of the at least one biomarker determined by the analyzing unit to the stored reference, wherein an amount of the at least one biomarker in the test sample which differs in comparison to the reference is indicative for the presence of bone disorder or wherein an essential identical amount of the at least one biomarker in the test sample in comparison to the reference is indicative for the absence of bone disorder.

Further, the present invention relates to a kit for diagnosing bone disorder comprising a detection agent for the at least one biomarker selected from any one of Tables 1a, 1b, 1c, 1d, 2a, 2b, 2c, or 2d and standards for the at least one biomarker the concentration of which is derived from a subject or a group of subjects known to suffer from bone disorder or derived from a subject or a group of subjects known to not suffer from bone disorder.

In particular, the present invention relates to a method for diagnosing impaired bone mineralization comprising:

(a) determining the amount of at least one biomarker selected from any one of Tables 1a, 1b, 1c, or 1d in a test sample of a subject suspected to suffer from impaired bone mineralization, and

(b) comparing the amounts determined in step (a) to a reference, whereby impaired bone mineralization is to be diagnosed.

In a particular embodiment of the method of the invention, a method is provided for diagnosing impaired bone mineralization comprising:

(a) selecting a male or female subject suspected to suffer from impaired bone mineralization;

(b) obtaining a test sample from said selected subject;

(c) pre-treating said sample in preparation for analysis;

(d) determining the amount of at least one biomarker selected from any one of Tables 1a, 1b, 1c, or 1d in said test sample, and

(e) comparing the amounts determined in step (d) to a reference; and

(f) based on the comparison of step (e), diagnose impaired bone mineralization by monitoring, confirmation or classification of the impaired bone mineralization or its symptoms.

In a preferred embodiment of the aforementioned method said subject has been brought into contact with a compound suspected to be capable of inducing impaired bone mineralization.

The present invention also relates to a method of determining whether a compound is capable of inducing impaired bone mineralization in a subject comprising:

(a) determining in a sample of a subject which has been brought into contact with a compound suspected to be capable of inducing impaired bone mineralization the amount of at least one biomarker selected from any one of Tables 1a, 1b, 1c, or 1d; and

(b) comparing the amounts determined in step (a) to a reference, whereby the capability of the compound to induce impaired bone mineralization is determined.

In a particular embodiment of the method of the invention, a method is provided for determining whether a compound is capable of inducing impaired bone mineralization in a subject comprising:

(a1) (i) selecting a male or female subject;

(ii) bringing said subject into contact with a compound suspected to be capable of inducing impaired bone mineralization, or

(a2) selecting a male or female subject brought into contact with a compound capable of inducing impaired bone mineralization;

(b) obtaining a test sample from said selected subject;

(c) pre-treating said sample in preparation for analysis;

(d) determining the amount of at least one biomarker selected from any one of Tables 1a, 1b, 1c, or 1d in said test sample, and

(e) comparing the amounts determined in step (d) to a reference; and

(f) based on the comparison of step (e), identifying whether the compound is capable of inducing impaired bone mineralization, or not.

In a preferred embodiment of the aforementioned method said compound is at least one compound selected from the group consisting of: Sodium perchlorate and Sodium fluoride.

In another preferred embodiment of the methods of the present invention said reference is derived from (i) a subject or group of subjects which suffers from impaired bone mineralization or (ii) a subject or group of subjects which has been brought into contact with at least one compound selected from the group consisting of: Sodium perchlorate and Sodium fluoride. In a more preferred embodiment of said method essentially identical amounts for the biomarkers in the test sample and the reference are indicative for impaired bone mineralization.

In another preferred embodiment of the methods of the present invention said reference is derived from (i) a subject or group of subjects known to not suffer from impaired bone mineralization or (ii) a subject or group of subjects which has not been brought into contact with at least one compound selected from the group consisting of: Sodium perchlorate and Sodium fluoride. In a more preferred embodiment of said methods amounts for the biomarkers which differ in the test sample in comparison to the reference are indicative for impaired bone mineralization.

In yet another embodiment of the methods of the present invention said reference is a calculated reference for the biomarkers for a population of subjects. In a more preferred embodiment of said methods amounts for the biomarkers which differ in the test sample in comparison to the reference are indicative for impaired bone mineralization.

The present invention also contemplates a method of identifying a substance for treating impaired bone mineralization comprising the steps of:

(a) determining in a sample of a subject suffering from impaired bone mineralization which has been brought into contact with a candidate substance suspected to be capable of treating impaired bone mineralization the amount of at least one biomarker selected from any one of Tables 1a, 1b, 1c, and 1d; and

(b) comparing the amounts determined in step (a) to a reference, whereby a substance capable of treating impaired bone mineralization is to be identified.

In a particular embodiment of the method of the invention, a method is provided for identifying a substance for treating impaired bone mineralization comprising:

(a1) (i) selecting a male or female subject;

(ii) bringing said subject into contact with a compound suspected to be capable of inducing impaired bone mineralization such that impaired bone mineralization is elicited, or

(a2) selecting a male or female suffering from impaired bone mineralization;

(b) obtaining a test sample from said selected subject;

(c) pre-treating said sample in preparation for analysis;

(d) determining the amount of at least one biomarker selected from any one of Tables 1a, 1b, 1c, or 1d in said test sample, and

(e) comparing the amounts determined in step (d) to a reference; and

(f) based on the comparison of step (e), identifying and selecting the substance for treating impaired bone mineralization.

In a preferred embodiment of the aforementioned method said reference is derived from (i) a subject or group of subjects which suffers from impaired bone mineralization or (ii) a subject or group of subjects which has been brought into contact with at least one compound selected from the group consisting of: Sodium perchlorate and Sodium fluoride. In a more preferred embodiment of said method amounts for the biomarkers which differ in the test sample and the reference are indicative for a substance capable of treating impaired bone mineralization.

In another preferred embodiment of the aforementioned method said reference is derived from (i) a subject or group of subjects known to not suffer from impaired bone mineralization or (ii) a subject or group of subjects which has not been brought into contact with at least one compound selected from the group consisting of: Sodium perchlorate and Sodium fluoride. In a more preferred embodiment of the said methods essentially identical amounts for the biomarkers in the test sample and the reference are indicative for a substance capable of treating impaired bone mineralization.

In yet another preferred embodiment of the aforementioned method said reference is a calculated reference for the biomarkers in a population of subjects. In a more preferred embodiment of the said methods essentially identical amounts for the biomarkers in the test sample and the reference are indicative for a substance capable of treating impaired bone mineralization.

The present invention also relates to the use of at least one biomarker selected from any one of Tables 1a, 1b, 1c, or 1d or a detection agent for the said biomarker for diagnosing impaired bone mineralization in a sample of a subject.

Moreover, the present invention relates to a device for diagnosing impaired bone mineralization in a sample of a subject suspected to suffer therefrom comprising:

(a) an analyzing unit comprising a detection agent for at least one biomarker selected from any one of Tables 1a, 1b, 1c, or 1d which allows for determining the amount of the said biomarker present in the sample; and, operatively linked thereto,

(b) an evaluation unit comprising a stored reference and a data processor which allows for comparing the amount of the said at least one biomarker determined by the analyzing unit to the stored reference, whereby impaired bone mineralization is diagnosed.

In a preferred embodiment of the device of the invention said stored reference is a reference derived from a subject or a group of subjects known to suffer from impaired bone mineralization or a subject or group of subjects which has been brought into contact with at least one compound selected from the group consisting of Sodium perchlorate and Sodium fluoride, and said data processor executes instructions for comparing the amount of the at least one biomarker determined by the analyzing unit to the stored reference, wherein an essentially identical amount of the at least one biomarker in the test sample in comparison to the reference is indicative for the presence of impaired bone mineralization or wherein an amount of the at least one biomarker in the test sample which differs in comparison to the reference is indicative for the absence of impaired bone mineralization.

In another preferred embodiment of the device of the invention said stored reference is a reference derived from a subject or a group of subjects known to not suffer from impaired bone mineralization or a subject or group of subjects which has not been brought into contact with at least one compound selected from the group consisting of Sodium perchlorate and Sodium fluoride, and said data processor executes instructions for comparing the amount of the at least one biomarker determined by the analyzing unit to the stored reference, wherein an amount of the at least one biomarker in the test sample which differs in comparison to the reference is indicative for the presence of impaired bone mineralization or wherein an essential identical amount of the at least one biomarker in the test sample in comparison to the reference is indicative for the absence of impaired bone mineralization.

Further, the present invention relates to a kit for diagnosing impaired bone mineralization comprising a detection agent for the at least one biomarker selected from any one of Tables 1a, 1b, 1c, and 1d and standards for the at least one biomarker the concentration of which is derived from a subject or a group of subjects known to suffer from impaired bone mineralization or derived from a subject or a group of subjects known to not suffer from impaired bone mineralization.

In particular, the present invention relates to a method for diagnosing inhibition of osteoblast activity comprising:

(a) determining the amount of at least one biomarker selected from any one of Tables 2a, 2b, 2c, and 2d in a test sample of a subject suspected to suffer from inhibition of osteoblast activity, and

(b) comparing the amounts determined in step (a) to a reference, whereby inhibition of osteoblast activity is to be diagnosed.

In a particular embodiment of the method of the invention, a method is provided for diagnosing inhibition of osteoblast activity comprising:

(a) selecting a male or female subject suspected to suffer from inhibition of osteoblast activity;

(b) obtaining a test sample from said selected subject;

(c) pre-treating said sample in preparation for analysis;

(d) determining the amount of at least one biomarker selected from any one of Tables 2a, 2b, 2c, or 2d in said test sample, and

(e) comparing the amounts determined in step (d) to a reference; and

(f) based on the comparison of step (e), diagnose inhibition of osteoblast activity by monitoring, confirmation or classification of the inhibition of osteoblast activity or its symptoms.

In a preferred embodiment of the aforementioned method said subject has been brought into contact with a compound suspected to be capable of inducing inhibition of osteoblast activity.

The present invention also relates to a method of determining whether a compound is capable of inducing inhibition of osteoblast activity in a subject comprising:

(a) determining in a sample of a subject which has been brought into contact with a compound suspected to be capable of inducing inhibition of osteoblast activity the amount of at least one biomarker selected from any one of Tables 2a, 2b, 2c, and 2d; and

(b) comparing the amounts determined in step (a) to a reference, whereby the capability of the compound to induce inhibition of osteoblast activity is determined.

In a particular embodiment of the method of the invention, a method is provided for determining whether a compound is capable of inducing inhibition of osteoblast activity in a subject comprising:

(a1) (i) selecting a male or female subject;

(ii) bringing said subject into contact with a compound suspected to be capable of inducing inhibition of osteoblast activity, or

(a2) selecting a male or female subject brought into contact with a compound capable of inducing inhibition of osteoblast activity;

(b) obtaining a test sample from said selected subject;

(c) pre-treating said sample in preparation for analysis;

(d) determining the amount of at least one biomarker selected from any one of Tables 2a, 2b, 2c, or 2d in said test sample, and

(e) comparing the amounts determined in step (d) to a reference; and

(f) based on the comparison of step (e), identifying whether the compound is capable of inducing inhibition of osteoblast activity, or not.

In a preferred embodiment of the aforementioned method said compound is at least one compound selected from the group consisting of: Calcitonin (Salmon) and Calcium carbonate.

In another preferred embodiment of the methods of the present invention said reference is derived from (i) a subject or group of subjects which suffers from inhibition of osteoblast activity or (ii) a subject or group of subjects which has been brought into contact with at least one compound selected from the group consisting of: Calcitonin (Salmon) and Calcium carbonate. In a more preferred embodiment of said method essentially identical amounts for the biomarkers in the test sample and the reference are indicative for inhibition of osteoblast activity.

In another preferred embodiment of the methods of the present invention said reference is derived from (i) a subject or group of subjects known to not suffer from inhibition of osteoblast activity or (ii) a subject or group of subjects which has not been brought into contact with at least one compound selected from the group consisting of: Calcitonin (Salmon) and Calcium carbonate. In a more preferred embodiment of said methods amounts for the biomarkers which differ in the test sample in comparison to the reference are indicative for inhibition of osteoblast activity.

In yet another embodiment of the methods of the present invention said reference is a calculated reference for the biomarkers for a population of subjects. In a more preferred embodiment of said methods amounts for the biomarkers which differ in the test sample in comparison to the reference are indicative for inhibition of osteoblast activity.

The present invention also contemplates a method of identifying a substance for treating inhibition of osteoblast activity comprising the steps of:

(a) determining in a sample of a subject suffering from inhibition of osteoblast activity which has been brought into contact with a candidate substance suspected to be capable of treating inhibition of osteoblast activity the amount of at least one biomarker selected from any one of Tables 2a, 2b, 2c, and 2d; and

(b) comparing the amounts determined in step (a) to a reference, whereby a substance capable of treating inhibition of osteoblast activity is to be identified.

In a particular embodiment of the method of the invention, a method is provided for identifying a substance for treating inhibition of osteoblast activity comprising:

(a1) (i) selecting a male or female subject;

(ii) bringing said subject into contact with a compound suspected to be capable of inducing inhibition of osteoblast activity such that inhibition of osteoblast activity is elicited, or

(a2) selecting a male or female suffering from inhibition of osteoblast activity;

(b) obtaining a test sample from said selected subject;

(c) pre-treating said sample in preparation for analysis;

(d) determining the amount of at least one biomarker selected from any one of Tables 2a, 2b, 2c, or 2d in said test sample, and

(e) comparing the amounts determined in step (d) to a reference; and

(f) based on the comparison of step (e), identifying and selecting the substance for treating inhibition of osteoblast activity.

In a preferred embodiment of the aforementioned method said reference is derived from (i) a subject or group of subjects which suffers from inhibition of osteoblast activity or (ii) a subject or group of subjects which has been brought into contact with at least one compound selected from the group consisting of: Calcitonin (Salmon) and Calcium carbonate. In a more preferred embodiment of said method amounts for the biomarkers which differ in the test sample and the reference are indicative for a substance capable of treating inhibition of osteoblast activity.

In another preferred embodiment of the aforementioned method said reference is derived from (i) a subject or group of subjects known to not suffer from inhibition of osteoblast activity or (ii) a subject or group of subjects which has not been brought into contact with at least one compound selected from the group consisting of: Calcitonin (Salmon) and Calcium carbonate. In a more preferred embodiment of the said methods essentially identical amounts for the biomarkers in the test sample and the reference are indicative for a substance capable of treating inhibition of osteoblast activity.

In yet another preferred embodiment of the aforementioned method said reference is a calculated reference for the biomarkers in a population of subjects. In a more preferred embodiment of the said methods essentially identical amounts for the biomarkers in the test sample and the reference are indicative for a substance capable of treating inhibition of osteoblast activity.

The present invention also relates to the use of at least one biomarker selected from any one of Tables 2a, 2b, 2c, or 2d or a detection agent for the said biomarker for diagnosing inhibition of osteoblast activity in a sample of a subject.

Moreover, the present invention relates to a device for diagnosing inhibition of osteoblast activity in a sample of a subject suspected to suffer therefrom comprising:

(a) an analyzing unit comprising a detection agent for at least one biomarker selected from any one of Tables 2a, 2b, 2c, or 2d which allows for determining the amount of the said biomarker present in the sample; and, operatively linked thereto,

(b) an evaluation unit comprising a stored reference and a data processor which allows for comparing the amount of the said at least one biomarker determined by the analyzing unit to the stored reference, whereby inhibition of osteoblast activity is diagnosed.

In a preferred embodiment of the device of the invention said stored reference is a reference derived from a subject or a group of subjects known to suffer from inhibition of osteoblast activity or a subject or group of subjects which has been brought into contact with at least one compound selected from the group consisting of Calcitonin (Salmon) and Calcium carbonate, and said data processor executes instructions for comparing the amount of the at least one biomarker determined by the analyzing unit to the stored reference, wherein an essentially identical amount of the at least one biomarker in the test sample in comparison to the reference is indicative for the presence of inhibition of osteoblast activity or wherein an amount of the at least one biomarker in the test sample which differs in comparison to the reference is indicative for the absence of inhibition of osteoblast activity.

In another preferred embodiment of the device of the invention said stored reference is a reference derived from a subject or a group of subjects known to not suffer from inhibition of osteoblast activity or a subject or group of subjects which has not been brought into contact with at least one compound selected from the group consisting of Calcitonin (Salmon) and Calcium carbonate, and said data processor executes instructions for comparing the amount of the at least one biomarker determined by the analyzing unit to the stored reference, wherein an amount of the at least one biomarker in the test sample which differs in comparison to the reference is indicative for the presence of inhibition of osteoblast activity or wherein an essential identical amount of the at least one biomarker in the test sample in comparison to the reference is indicative for the absence of inhibition of osteoblast activity.

Further, the present invention relates to a kit for diagnosing inhibition of osteoblast activity comprising a detection agent for the at least one biomarker selected from any one of Tables 2a, 2b, 2c, or 2d and standards for the at least one biomarker the concentration of which is derived from a subject or a group of subjects known to suffer from inhibition of osteoblast activity or derived from a subject or a group of subjects known to not suffer from inhibition of osteoblast activity.

In particular the present invention contemplates also the following specific methods, uses, devices and kits.

The following definitions and explanations apply mutatis mutandis to all the previous embodiments of the present invention as well as the embodiments described in the following.

The methods referred to in accordance with the present invention may essentially consist of the aforementioned steps or may include further steps. Further steps may relate to sample pre-treatment or evaluation of the diagnostic results obtained by the methods. Preferred further evaluation steps are described elsewhere herein. The methods may partially or entirely be assisted by automation. For example, steps pertaining to the determination of the amount of a biomarker can be automated by robotic and automated reader devices. Likewise, steps pertaining to a comparison of amounts can be automated by suitable data processing devices, such as a computer, comprising a program code which when being executed carries out the comparison automatically. A reference in such a case will be provided from a stored reference, e.g., from a database. It is to be understood that the method is, preferably, a method carried out ex viva on a sample of a subject, i.e. not practised on the human or animal body.

The term “diagnosing” as used herein refers to assessing the probability according to which a subject is suffering from a condition, such as intoxication, disease or disorder referred to herein, or has a predisposition for such a condition. Diagnosis of a predisposition may sometimes be referred to as prognosis or prediction of the likelihood that a subject will develop the condition within a predefined time window in the future. As will be understood by those skilled in the art, such an assessment, although preferred to be, may usually not be correct for 100% of the subjects to be diagnosed. The term, however, requires that a statistically significant portion of subjects can be identified as suffering from the condition or having a predisposition for the condition. Whether a portion is statistically significant can be determined without further ado by the person skilled in the art using various well known statistic evaluation tools, e.g., determination of confidence intervals, p-value determination, Student's t-test, Mann-Whitney test, etc. Details are found in Dowdy and Wearden, Statistics for Research, John Wiley & Sons, New York 1983. Preferred confidence intervals are at least 50%, at least 60%, at least 70%, at least 80%, at least 90% or at least 95%. The p-values are, preferably, 0.2, 0.1, 0.05.

Diagnosing according to the present invention also includes monitoring, confirmation, and classification of a condition or its symptoms as well as a predisposition therefor. Monitoring refers to keeping track of an already diagnosed condition or predisposition. Monitoring encompasses, e.g., determining the progression of the condition or predisposition, determining the influence of a particular treatment on the progression of the condition or the influence of prophylactic measures such as a prophylactic treatment or diet on the development of the condition in a subject having a predisposition. Said treatment, prophylactic measure or diet may be adjusted and the influence of the adjustment may be investigated as an aspect of the monitoring. Moreover, if progression of the condition or a predisposition therefor is monitored, said monitoring may also include determining a monitoring frequency and to recommend and/or carry out additional monitoring measures such as measurement of additional biochemical or other health parameters. Confirmation relates to the strengthening or substantiating a diagnosis of the condition or a predisposition for the condition already determined using other indicators or markers. Confirmation may also include in an aspect the administration or adaptation of therapeutic measures based on the confirmed condition or predisposition therefor. Classification relates to (i) allocating the condition into different classes, e.g., corresponding to the strength of the symptoms accompanying the condition, or (ii) differentiating between different stages, disease or disorders accompanying the condition. Classification may also include in an aspect the administration or adaptation of therapeutic measures based on the classified condition, symptoms or predisposition therefor. A predisposition for the condition can be classified based on the degree of the risk, i.e. the probability according to which a subject will develop the condition later. Moreover, classification also, preferably, includes allocating a mode of action to a compound to be tested by the methods of the present invention. Specifically, the methods of the present invention allow for determination of a specific mode of action of a compound for which such mode of action is not yet known. This is, preferably, achieved by comparing the amount determined for the at least one biomarker or a biomarker profile representative for said compound to the amount of the biomarker or biomarker profile determined for a compound for which the mode of action is known as a reference. The classification of the mode of action allows an even more reliable assessment of toxicity of a compound because the molecular targets of the compound are identified. The methods of the present invention aiming at diagnosing a disease or condition may be used for screening compounds for toxicological effects and reporting thereon as well as in compound development, e.g., in increasing safety or in developing drugs or identifying effective concentrations.

In accordance with the present invention, a compound can also be identified as being capable of inducing a bone disorder. Such identification, preferably, also includes making suggestions for the manufacture, handling, storage and/or transport of the compound and its applications. Such suggestions include establishing safety protocols for manufacture, handling, storage, transport and/or application, labelling the compound according to its toxicity potential, limiting exposure to humans, animals and/or to the environment. Moreover, if a compound is identified as eliciting a bone disorder, safety levels such as LD50/LC50 and/or ED50/EC50 values and derived thresholds are, preferably, determined.

The term “bone disorder” as used herein relates to any disorder of the bone affecting the compact or spongy bone tissue. Bone tissue as used herein, preferably, is a ridgid type of connective tissue and cartilage with inclusions of minerals, in particular, carbonated hydroxyapetite. Most bones comprise compact bone tissue forming a dense outer shaft as well as trabecullar bone tissue which forms an inner spongy network of bone rod- and plate-like structures. Three cell types are pivotally invloved in the formation of bones, i.e. osteoblasts, osteocytes and osteoblasts. Osteoblasts are mesenchymal precursor cells of osteocytes. Osteocytes are mature cells in the mineralized bone tissue. Osteoclasts are macrophage-like cells which are caopable of demineralizing mineralized bone tissue (bone resorption) and which are, therefore, involved in bone growth and restructuration. Mineralization processes within the growing bones as well as the mature bones are goverend by various endogenous and exogenous factors. An impaired mineralization process, either due to impaired osteblast maturation or activity, impaired mineralization and/or impaired osteoclast activity results in various bone disorders also called osteopathies. The bone disorder referred to herein is, preferably, accompanioed by impaired mineralization and/or inhibition of osteoblast activity and/or maturation. A preferred and prominent bone disorder with impaired mineralization is osteoporosis. Osteoporosis is associated with a reduced bone mineral density (BMD) and deteriorated bone architecture. Another preferred disorder with impaired mineralization is osteopenia. Osteopenia is also accompanied with a reduced BMD. Moreover, further bone metabolic disorders with impaired mineralization include Padget's disease of the bones, osteomalacia, hypophosphatasia, or osteitis fibrosa cystica. Bone disorders as referred to herein encompass impaired bone mineralization and the symptoms and consequences associated therewith as well as inhibition of osteoblast activity in the bones.

Preferably, a bone disorder as used herein is induced by or is the result of the administration of a chemical compound or drug, i.e. so-called toxin-induced bone disorder.

The symptoms and clinical signs of the aforementioned manifestations of bone disorder are well known to the person skilled in the art and are described in detail in standard books of toxicology, e.g., H. Marquardt, S. G. Schäfer, R. O. McClellan, F. Welsch (eds.), “Toxicology”, Chapter 13: The Liver, 1999, Academic Press, London.

Preferably, the at least one biomarker to be determined by the methods of the present invention is selected from any one of Tables 1a, 1b, 1c, and 1d if the bone disorder is accompanied by impaired mineralization.

Also preferably, the at least one biomarker to be determined by the methods of the present invention is selected from any one of Tables 2a, 2b, 2c, and 2d if the bone disorder is accompanied by inhibition of osteoblast activity and/or maturation.

It was found in accordance with the present invention that a combination of more than one of the biomarkers listed in the Tables further strengthen the diagnosis since each of the biomarkers is an apparently statistically independent predictor for the diagnosis. Moreover, the specificity for bone disorder is also significantly increased since influences from other tissues on the marker abundance are counterbalanced. Thus, the term “at least one” as used herein, preferably, refers to a combination of at least 2, at least 3, at least 4, at least 5, at least 6, at least 7, at least 8, at least 9 or at least 10 of the biomarkers referred to in any one of the accompanying Tables. Preferably, all biomarkers recited in any one of the Tables are to be determined in combination in accordance with the methods of the present invention.

In the following, preferred biomarkers and groups of biomarkers are specified for the bone disorders and subjects referred to in the Tables, below.

Tables 1a and 1b (impaired Bone mineralisation): Phenylalanine, myo-Inositol-2-phosphate, Lysophosphatidylcholine (C17:0), Eicosaenoic acid (C20:1) No 02 or Glucose.

Tables 1c and 1d (impaired Bone mineralisation): TAG No 07, Pyruvate, Heptadecanoic acid (C17:0), TAG (C18:1, C18:2) or Hippuric acid.

Tables 2a and 2b (Bone osteoblast inhibitors): Isoleucine, Ketoleucine, Leucine, Dodecanol or Phosphate (inorganic and organic phosphates).

Tables 2c and 2d (Bone osteoblast inhibitors): TAG (C16:0, C18:1, C18:3), Cholesterolester No 01, Threonine, 18-Hydroxy-1′-deoxycorticosterone or TAG (C16:0, C18:2).

Thus, preferably, the at least one biomarker is at least one biomarker selected from the aforementioned group or the at least one biomarker is a combination of biomarkers consisting or comprising the aforementioned group of biomarkers. The aforementioned biomarkers and combinations of biomarkers have been identified as key biomarkers having a particular high diagnostic value as described in more detail in the accompanying Examples.

Furthermore, other biomarkers or clinical parameters including known metabolites, genetic mutations, transcript and/or protein amounts or enzyme activities may still be determined in addition. Such, additional clinical or biochemical parameters which may be determined in accordance with the method of the present invention are well known in the art.

The term “biomarker” as used herein refers to a chemical compound whose presence or concentration in a sample is indicative for the presence or absence or strength of a condition, preferably, bone disorder as referred to herein. The chemical compound is, preferably, a metabolite or an analyte derived therefrom. An analyte is a chemical compound which can be identical to the actual metabolite found in an organism. However, the term also includes derivatives of such metabolites which are either endogenously generated or which are generated during the isolation or sample pre-treatment or as a result of carrying out the methods of the invention, e.g., during the purification and/or determination steps. In specific cases the analyte is further characterized by chemical properties such as solubility. Due to the said properties, the analyte may occur in polar or lipid fractions obtained during the purification and/or determination process. Thus, chemical properties and, preferably, the solubility shall result in the occurrence of an analyte in either polar or lipid fractions obtained during the purification and/or determination process. Accordingly, the said chemical properties and, in particular the solubility taken into account as the occurrence of an analyte in either polar or lipid fractions obtained during the purification and/or determination process shall further characterize the analyte and assist in its identification. Details on how these chemical properties can be determined and taken into account are found in the accompanying Examples described below. Preferably, the analyte represents the metabolite in a qualitative and quantitative manner and, thus, allows inevitably concluding on the presence or absence or the amount of the metabolite in a subject or at least in the test sample of said subject. Biomarker, analyte and metabolite are referred to herein in the singular but also include the plurals of the terms, i.e. refer to a plurality of biomarker, analyte or metabolite molecules of the same molecular species. Moreover, a biomarker according to the present invention is not necessarily corresponding to one molecular species. Rather, the biomarker may comprise stereoisomers or enantiomers of a compound. Further, a biomarker can also represent the sum of isomers of a biological class of isomeric molecules. Said isomers shall exhibit identical analytical characteristics in some cases and are, therefore, not distinguishable by various analytical methods including those applied in the accompanying Examples described below. However, the isomers will share at least identical sum formula parameters and, thus, in the case of, e.g., lipids an identical chain length and identical numbers of double bonds in the fatty acid and/or sphingo base moieties

The term “test sample” as used herein refers to samples to be used for the diagnosis of bone disorder by the methods of the present invention. Preferably, said test sample is a biological sample. Samples from biological sources (i.e. biological samples) usually comprise a plurality of metabolites. Preferred biological samples to be used in the method of the present invention are samples from body fluids, preferably, blood, plasma, serum, saliva, bile, urine or cerebrospinal fluid, or samples derived, e.g. by biopsy, from cells, tissues or organs, preferably from the liver. More preferably, the sample is a blood, plasma or serum sample, most preferably, a plasma sample. Biological samples are derived from a subject as specified elsewhere herein. Techniques for obtaining the aforementioned different types of biological samples are well known in the art. For example, blood samples may be obtained by blood taking while tissue or organ samples are to be obtained, e.g. by biopsy.

The aforementioned samples are, preferably, pre-treated before they are used for the methods of the present invention. As described in more detail below, said pre-treatment may include treatments required to release or separate the compounds or to remove excessive material or waste. Suitable techniques comprise centrifugation, extraction, fractioning, ultra-filtration, protein precipitation followed by filtration and purification and/or enrichment of compounds. Moreover, other pretreatments are carried out in order to provide the compounds in a form or concentration suitable for compound analysis. For example, if gas-chromatography coupled mass spectrometry is used in the method of the present invention, it will be required to derivatize the compounds prior to the said gas chromatography. Suitable and necessary pre-treatments depend on the means used for carrying out the method of the invention and are well known to the person skilled in the art. Pre-treated samples as described before are also comprised by the term “sample” as used in accordance with the present invention.

The term “subject” as used herein relates to animals, preferably to mammals such as mice, rats, guinea pigs, rabbits, hamsters, pigs, sheep, dogs, cats, horses, monkeys, or cows and, also preferably, to humans. More preferably, the subject is a rodent and, most preferably, a rat. Other animals which may be diagnosed applying the methods of the present invention are fishes, birds or reptiles. Preferably, said subject was in or has been brought into contact with a compound suspected to be capable of inducing bone disorder. A subject which has been brought into contact with a compound suspected to induce bone disorder may, e.g., be a laboratory animal such as a rat which is used in a screening assay for, e.g., toxicity of compounds. A subject suspected to have been in contact with a compound capable of inducing bone disorder may be also a subject to be diagnosed for selecting a suitable therapy. Preferably, a compound capable of inducing bone disorder as used herein is Sodium perchlorate, Sodium fluoride, Calcitonin (Salmon) and Calcium carbonate.

Preferably, the at least one biomarker to be determined by the methods of the present invention is selected from any one of Tables 1a or 1b if the subject is a female.

Preferably, the at least one biomarker to be determined by the methods of the present invention is selected from any one of Tables 1c, 1d, 2a, 2b, 2c, or 2d if the subject is a male.

The term “determining the amount” as used herein refers to determining at least one characteristic feature of the biomarker, i.e. the metabolite or analyte. Characteristic features in accordance with the present invention are features which characterize the physical and/or chemical properties including biochemical properties of a biomarker. Such properties include, e.g., molecular weight, viscosity, density, electrical charge, spin, optical activity, colour, fluorescence, chemoluminescence, elementary composition, chemical structure, capability to react with other compounds, capability to elicit a response in a biological read out system (e.g., induction of a reporter gene) and the like. Values for said properties may serve as characteristic features and can be determined by techniques well known in the art. Moreover, the characteristic feature may be any feature which is derived from the values of the physical and/or chemical properties of a biomarker by standard operations, e.g., mathematical calculations such as multiplication, division or logarithmic calculus. Most preferably, the at least one characteristic feature allows the determination and/or chemical identification of the biomarker and its amount. Accordingly, the characteristic value, preferably, also comprises information relating to the abundance of the biomarker from which the characteristic value is derived. For example, a characteristic value of a biomarker may be a peak in a mass spectrum. Such a peak contains characteristic information of the biomarker, i.e. the m/z (mass to charge ratio) information, as well as an intensity value being related to the abundance of the said biomarker (i.e. its amount) in the sample.

As discussed before, the at least one biomarker to be determined in accordance with the methods of the present invention may be, preferably, determined quantitatively or semi-quantitatively. For quantitative determination, either the absolute or precise amount of the biomarker will be determined or the relative amount of the biomarker will be determined based on the value determined for the characteristic feature(s) referred to herein above. The relative amount may be determined in a case were the precise amount of a biomarker can or shall not be determined. In said case, it can be determined whether the amount in which the biomarker is present is enlarged or diminished with respect to a second sample comprising said biomarker in a second amount. Quantitatively analysing a biomarker, thus, also includes what is sometimes referred to as semi-quantitative analysis of a biomarker.

Moreover, determining as used in the methods of the present invention, preferably, includes using a compound separation step prior to the analysis step referred to before. Preferably, said compound separation step yields a time resolved separation of the at least one biomarker comprised by the sample. Suitable techniques for separation to be used preferably in accordance with the present invention, therefore, include all chromatographic separation techniques such as liquid chromatography (LC), high performance liquid chromatography (HPLC), gas chromatography (GC), thin layer chromatography, size exclusion or affinity chromatography. These techniques are well known in the art and can be applied by the person skilled in the art without further ado. Most preferably, LC and/or GC are chromatographic techniques to be envisaged by the methods of the present invention. Suitable devices for such determination of biomarkers are well known in the art. Preferably, mass spectrometry is used in particular gas chromatography mass spectrometry (GC-MS), liquid chromatography mass spectrometry (LC-MS), direct infusion mass spectrometry or Fourier transform ion-cyclotrone-resonance mass spectrometry (FT-ICR-MS), capillary electrophoresis mass spectrometry (CE-MS), high-performance liquid chromatography coupled mass spectrometry (HPLC-MS), quadrupole mass spectrometry, any sequentially coupled mass spectrometry, such as MS-MS or MS-MS-MS, inductively coupled plasma mass spectrometry (ICP-MS), pyrolysis mass spectrometry (Py-MS), ion mobility mass spectrometry or time of flight mass spectrometry (TOF). Most preferably, LC-MS and/or GC-MS are used as described in detail below. Said techniques are disclosed in, e.g., Nissen 1995, Journal of Chromatography A, 703: 37-57, U.S. Pat. No. 4,540,884 or U.S. Pat. No. 5,397,894, the disclosure content of which is hereby incorporated by reference. As an alternative or in addition to mass spectrometry techniques, the following techniques may be used for compound determination: nuclear magnetic resonance (NMR), magnetic resonance imaging (MRI), Fourier transform infrared analysis (FT-IR), ultraviolet (UV) spectroscopy, refraction index (RI), fluorescent detection, radiochemical detection, electrochemical detection, light scattering (LS), dispersive Raman spectroscopy or flame ionisation detection (FID). These techniques are well known to the person skilled in the art and can be applied without further ado. The method of the present invention shall be, preferably, assisted by automation. For example, sample processing or pre-treatment can be automated by robotics. Data processing and comparison is, preferably, assisted by suitable computer programs and databases. Automation as described herein before allows using the method of the present invention in high-throughput approaches.

Moreover, the biomarker can also be determined by a specific chemical or biological assay. Said assay shall comprise means which allow for specifically detecting the biomarker in the sample. Preferably, said means are capable of specifically recognizing the chemical structure of the biomarker or are capable of specifically identifying the biomarker based on its capability to react with other compounds or its capability to elicit a response in a biological read out system (e.g., induction of a reporter gene). Means which are capable of specifically recognizing the chemical structure of a biomarker are, preferably, detection agents which specifically bind to the biomarker, more preferably, antibodies or other proteins which specifically interact with chemical structures, such as receptors or enzymes, or aptameres. Specific antibodies, for instance, may be obtained using the biomarker as antigen by methods well known in the art. Antibodies as referred to herein include both polyclonal and monoclonal antibodies, as well as fragments thereof, such as Fv, Fab and F(ab)₂ fragments that are capable of binding the antigen or hapten. The present invention also includes humanized hybrid antibodies wherein amino acid sequences of a non-human donor antibody exhibiting a desired antigen-specificity are combined with sequences of a human acceptor antibody. Moreover, encompassed are single chain antibodies. The donor sequences will usually include at least the antigen-binding amino acid residues of the donor but may comprise other structurally and/or functionally relevant amino acid residues of the donor antibody as well. Such hybrids can be prepared by several methods well known in the art. Suitable proteins which are capable of specifically recognizing the metabolite are, preferably, enzymes which are involved in the metabolic conversion of the said biomarker. Said enzymes may either use the biomarker, e.g., a metabolite, as a substrate or may convert a substrate into the biomarker, e.g., metabolite. Moreover, said antibodies may be used as a basis to generate oligopeptides which specifically recognize the biomarker. These oligopeptides shall, for example, comprise the enzyme's binding domains or pockets for the said biomarker. Suitable antibody and/or enzyme based assays may be RIA (radioimmunoassay), ELISA (enzyme-linked immunosorbent assay), sandwich enzyme immune tests, electrochemiluminescence sandwich immunoassays (ECLIA), dissociation-enhanced lanthanide fluoro immuno assay (DELFIA) or solid phase immune tests. Aptameres which specifically bind to the biomarker can be generated by methods well known in the art (Ellington 1990, Nature 346:818-822; Vater 2003, Curr Opin Drug Discov Devel 6(2): 253-261). Moreover, the biomarker may also be identified based on its capability to react with other compounds, i.e. by a specific chemical reaction. Further, the biomarker may be determined in a sample due to its capability to elicit a response in a biological read out system. The biological response shall be detected as read out indicating the presence and/or the amount of the metabolite comprised by the sample. The biological response may be, e.g., the induction of gene expression or a phenotypic response of a cell or an organism.

The term “reference” refers to values of characteristic features of the at least one biomarker and, preferably, values indicative for an amount of the said biomarker which can be correlated to bone disorder.

Such references are, preferably, obtained from a sample derived from a subject or group of subjects which suffer from bone disorder or from a sample derived from a subject or group of subjects which have/has been brought into contact with Sodium perchlorate, Sodium fluoride, Calcitonin (Salmon) or Calcium carbonate. A subject or group of subjects may be brought into contact with the said compounds by each topic or systemic administration mode as long as the compounds become bioavailable.

Preferably, the aforementioned compounds can be administered to the subject or the indiividuals of the group of subjects from which the reference is derived as described in the accompanying Examples and Tables below.

In particular, Sodium perchlorate or Sodium fluoride as referred to herein are compounds capable of inducing impaired mineralization while Calcitonin (Salmon) and Calcium carbonate shall be capable of inducing inhibition of osteoblast activity and/or maturation.

Alternatively, but nevertheless also preferred, the reference may be obtained from sample derived from a subject or group of subjects which has not been brought into contact with Sodium perchlorate, Sodium fluoride, Calcitonin (Salmon) or Calcium carbonate or a healthy subject or group of such subjects with respect to bone disorder and, more preferably, other diseases as well.

The reference may be determined as described hereinabove for the amounts of the biomarkers. In particular, a reference is, preferably, obtained from a sample of a group of subjects as referred to herein by determining the relative or absolute amounts of each of the at least one biomarker(s) in samples from each of the individuals of the group separately and subsequently determining a median or average value for said relative or absolute amounts or any parameter derived therefrom by using statistical techniques referred to elsewhere herein. Alternatively, the reference may be, preferably, obtained by determining the relative or absolute amount for each of the at least one biomarker in a sample from a mixture of samples of the group of subjects as referred to herein. Such a mixture, preferably, consists of portions of equal volume from samples obtained from each of the individuals of the said group.

Moreover, the reference, also preferably, could be a calculated reference, most preferably the average or median value, for the relative or absolute amount for each of the at least one biomarker derived from a population of individuals. Said population of individuals is the population from which the subject to be investigated by the method of the present invention originates. However, it is to be understood that the population of subjects to be investigated for determining a calculated reference, preferably, either consist of apparently healthy subjects (e.g. untreated) or comprise a number of apparently healthy subjects which is large enough to be statistically resistant against significant average or median changes due to the presence of the test subject(s) in the said population. The absolute or relative amounts of the at least one biomarker of said individuals of the population can be determined as specified elsewhere herein. How to calculate a suitable reference value, preferably, the average or median, is well known in the art. Other techniques for calculating a suitable reference include optimization using receiver operating characteristics (ROC) curve calculations which are also well known in the art and which can be performed for an assay system having a given specificity and sensitivity based on a given cohort of subjects without further ado. The population or group of subjects referred to before shall comprise a plurality of subjects, preferably, at least 5, 10, 50, 100, 1,000 or 10,000 subjects up to the entire population. More preferably, the group of subjects referred to in this context is a group of subjects having a size being statistically representative for a given population, i.e. a statistically representative sample. It is to be understood that the subject to be diagnosed by the methods of the present invention and the subjects of the said plurality of subjects are of the same species and, preferably, of the same gender.

More preferably, the reference will be stored in a suitable data storage medium such as a database and are, thus, also available for future diagnoses. This also allows efficiently diagnosing predisposition for bone disorder because suitable reference results can be identified in the database once it has been confirmed (in the future) that the subject from which the corresponding reference sample was obtained (indeed) developed bone disorder.

The term “comparing” refers to assessing whether the amount of the qualitative or quantitative determination of the at least one biomarker is identical to a reference or differs therefrom.

In case the reference results are obtained from a sample derived from a subject or group of subjects suffering from bone disorder or a subject or group of subjects which has been brought into contact with Sodium perchlorate, Sodium fluoride, Calcitonin (Salmon) or Calcium carbonate, bone disorder can be diagnosed based on the degree of identity or similarity between the amounts obtained from the test sample and the aforementioned reference, i.e. based on an identical qualitative or quantitative composition with respect to the at least one biomarker. Identical amounts include those amounts which do not differ in a statistically significant manner and are, preferably, within at least the interval between 1st and 99th percentile, 5th and 95th percentile, 10th and 90th percentile, 20th and 80th percentile, 30th and 70th percentile, 40th and 60th percentile of the reference, more preferably, the 50th, 60th, 70th, 80th, 90th or 95th percentile of the reference. A reference obtained from a sample derived from a subject or group of subjects suffering from bone disorder or a subject or group of subjects which has been brought into contact with Sodium perchlorate, Sodium fluoride, Calcitonin (Salmon) or Calcium carbonate, can be applied in the methods of the present invention in order to diagnose bone disorder or for determining whether a compound is capable of inducing bone disorder in a subject. In such a case, preferably, an amount of the at least one biomarker which is essentially identical to the reference will be indicative for the presence of bone disorder or a compound which is capable of inducing bone disorder, while an amount of the at least one biomarker which differs from the reference will be indicative for the absence of bone disorder or a compound which is not capable of inducing bone disorder.

Moreover, a reference obtained from a sample derived from a subject or group of subjects suffering from bone disorder or a subject or group of subjects which has been brought into contact with Sodium perchlorate, Sodium fluoride, Calcitonin (Salmon) or Calcium carbonate, can be applied for identifying a substance for treating bone disorder. In such a case, preferably, an amount of the at least one biomarker which differs from the reference will be indicative for a substance suitable for treating bone disorder, while an amount of the at least one biomarker which is essentially identical to the reference will be indicative for a substance which is not capable of treating bone disorder.

In case the reference results are obtained from a sample of a subject or group of subjects which has not been brought into contact with Sodium perchlorate, Sodium fluoride, Calcitonin (Salmon) or Calcium carbonate or which does not suffer from bone disorder, said bone disorder can be diagnosed based on the differences between the test amounts obtained from the test sample and the aforementioned reference, i.e. differences in the qualitative or quantitative composition with respect to the at least one biomarker.

The same applies if a calculated reference as specified above is used.

The difference may be an increase in the absolute or relative amount of the at least one biomarker (sometimes referred to as up-regulation of the biomarker; see also Examples) or a decrease in either of said amounts or the absence of a detectable amount of the biomarker (sometimes referred to as down-regulation of the biomarker; see also Examples). Preferably, the difference in the relative or absolute amount is significant, i.e. outside of the interval between 45th and 55th percentile, 40th and 60th percentile, 30th and 70th percentile, 20th and 80th percentile, 10th and 90th percentile, 5th and 95th percentile, 1st and 99th percentile of the reference.

A reference obtained from a sample derived from a subject or group of subjects which has not been brought into contact with Sodium perchlorate, Sodium fluoride, Calcitonin (Salmon) or Calcium carbonate or which does not suffer from bone disorder can be applied in the methods of the present invention in order to diagnose the bone disorder or for determining whether a compound is capable of inducing bone disorder in a subject. In such a case, preferably, an amount of the at least one biomarker which differs from the reference will be indicative for the presence of bone disorder or a compound which is capable of inducing bone disorder, while an amount of the at least one biomarker which is essentially identical to the reference will be indicative for the absence of bone disorder or a compound which is not capable of inducing bone disorder. Moreover, a reference obtained from a sample derived from a subject or group of subjects which has not been brought into contact with Sodium perchlorate, Sodium fluoride, Calcitonin (Salmon) or Calcium carbonate, or which does not suffer from bone disorder can be applied for identifying a substance for treating bone disorder. In such a case, preferably, an amount of the at least one biomarker which is essentially identical to the reference will be indicative for a substance suitable for treating bone disorder, while an amount of the at least one biomarker which differs from the reference will be indicative for a substance which is not suitable for treating bone disorder.

Preferred references are those referred to in the accompanying Tables or those which can be generated following the accompanying Examples. Moreover, relative differences, i.e. increases or decreases in the amounts for individual biomarkers, are preferably, those recited in the Tables below. Moreover, preferably, the extent of an observed difference, i.e. an increase or decrease, is preferably, an increase or decrease according to the factor indicated in the Tables, below.

Preferably, the at least one biomarker when selected from Tables 1a, 1c, 2a, or 2c is increased with respect to a reference obtained from a sample derived from a subject or group of subjects which has not been brought into contact with Sodium perchlorate, Sodium fluoride, Calcitonin (Salmon) or Calcium carbonate or a sample obtained from a healthy subject or group of subjects as indicated in the said Tables.

Preferably, the at least one biomarker when selected from Tables 1b, 1d, 2b or 2d decreased with respect to a reference obtained from a sample derived from a subject or group of subjects which has not been brought into contact with Sodium perchlorate, Sodium fluoride, Calcitonin (Salmon) or Calcium carbonate or a sample obtained from a healthy subject or group of subjects as indicated in the said Tables.

The comparison is, preferably, assisted by automation. For example, a suitable computer program comprising algorithm for the comparison of two different data sets (e.g., data sets comprising the values of the characteristic feature(s)) may be used. Such computer programs and algorithm are well known in the art. Notwithstanding the above, a comparison can also be carried out manually.

The term “substance for treating bone disorder” refers to compounds which may directly interfere with the biological mechanisms inducing bone disorder referred to elsewhere in this specification. Alternatively, but also preferred the compounds may interfere with the development or progression of symptoms associated with the bone disorder. Substances to be identified by the method of the present invention may be organic and inorganic chemicals, such as small molecules, polynucleotides, oligonucleotides including siRNA, ribozymes or micro RNA molecules, peptides, polypeptides including antibodies or other artificial or biological polymers, such as aptameres. Preferably, the substances are suitable as drugs, pro-drugs or lead substances for the development of drugs or pro-drugs. Thus, in an aspect of the invention, the method may further include a step comprising identifying and/or confirming the identified and selected substance a drug, pro-drug or drug or pro-drug candidate for further clinical development. Such clinical development may, preferably, includes pharmacological studies of the substance, toxicological determinations of the substance, animal and human drug testing, including clinical trials of all phases.

It is to be understood that if the methods of the present invention are to be used for identifying drugs for the therapy of bone disorder or for toxicological assessments of compounds (i.e. determining whether a compound is capable of inducing bone disorder), test samples of a plurality of subjects may be investigated for statistical reasons. Preferably, the metabolome within such a cohort of test subjects shall be as similar as possible in order to avoid differences which are caused, e.g., by factors other than the compound to be investigated. Subjects to be used for the said methods are, preferably, laboratory animals such as rodents and more preferably rats. It is to be understood further that the said laboratory animals shall be, preferably, sacrificed after completion of the methods of the present invention. All subjects of a cohort test and reference animals shall be kept under identical conditions to avoid any differential environmental influences. Suitable conditions and methods of providing such animals are described in detail in WO2007/014825. Said conditions are hereby incorporated by reference.

Accordingly, the methods of the invention aiming at identifying a substance for treating bone disorders and, in particular, impaired bone mineralization and the symptoms and consequences associated therewith and/or inhibition of osteoblast activity, preferably, include additional steps. Prefersbly, further steps include carrying out preclinical studies with the substance in order to identify pharmacological and/or toxicological parameters thereof, such as ED50/EC50 and/or LD50/LC50 thresholds, carrying out clinical trials, e.g., for determining therapeutic efficacy and safety of the substance and the formulation of the identified substance in a pharmaceutically acceptable form.

The substance can, preferably, be formulated for topical or systemic administration. Conventionally, a drug will be administered intra-muscular or, subcutaneous. However, depending on the nature and the mode of action of a substance, it may, however, be administered by other routes as well. The substance is, preferably, formulated for administration in conventional dosage forms and prepared by combining the identified substance with standard pharmaceutical carriers according to conventional procedures. These procedures may involve mixing, granulating, and compression, or dissolving the ingredients as appropriate to the desired preparation. It will be appreciated that the form and character of the pharmaceutical acceptable carrier or diluent is dictated by the amount of active ingredient with which it is to be combined, the route of administration, and other well-known variables. A carrier must be acceptable in the sense of being compatible with the other ingredients of the formulation and being not deleterious to the recipient thereof. The pharmaceutical carrier employed may include a solid, a gel, or a liquid. Without being limiting, examples for solid carriers are lactose, terra alba, sucrose, talc, gelatin, agar, pectin, acacia, magnesium stearate, stearic acid and the like. Without being limiting, exemplary of liquid carriers are phosphate buffered saline solution, syrup, oil, water, emulsions, various types of wetting agents, and the like. Similarly, the carrier or diluent may include time delay material well known to the art, such as glyceryl mono-stearate or glyceryl distearate alone or with a wax. Said suitable carriers comprise those mentioned above and others well known in the art, see, e.g., Remington's Pharmaceutical Sciences, Mack Publishing Company, Easton, Pa. A diluent is selected so as not to affect the biological activity of the combination. Without being limiting, examples of such diluents are distilled water, physiological saline, Ringer's solutions, dextrose solution, and Hank's solution. In addition, the pharmaceutical composition or formulation may also include other carriers, adjuvants, or non-toxic, non-therapeutic, non-immunogenic stabilizers and the like. It is to be understood that the formulation of a substance as a drug takes place under GMP standardized conditions or the like in order to ensure quality, pharmaceutical security, and effectiveness.

The methods of the present invention can be, preferably, implemented by the device of the present invention. A device as used herein shall comprise at least the aforementioned units. The units of the device are operatively linked to each other. How to link the units in an operating manner will depend on the type of units included into the device. For example, where means for automatically qualitatively or quantitatively determining the at least one biomarker are applied in an analyzing unit, the data obtained by said automatically operating unit can be processed by the evaluation unit, e.g., by a computer program which runs on a computer being the data processor in order to facilitate the diagnosis. Preferably, the units are comprised by a single device in such a case. However, the analyzing unit and the evaluation unit may also be physically separate. In such a case operative linkage can be achieved via wire and wireless connections between the units which allow for data transfer. A wireless connection may use Wireless LAN (WLAN) or the internet. Wire connections may be achieved by optical and non-optical cable connections between the units. The cables used for wire connections are, preferably, suitable for high throughput data transport

A preferred analyzing unit for determining at least one biomarker comprises a detection agent, such as an antibody, protein or aptamere which specifically recognizes the at least one biomarker as specified elsewhere herein, and a zone for contacting said detection agent with the sample to be tested. The detection agent may be immobilized on the zone for contacting or may be applied to the said zone after the sample has been loaded. The analyzing unit shall be, preferably, adapted for qualitatively and/or quantitatively determine the amount of complexes of the detection agent and the at least one biomarker. It will be understood that upon binding of the detection agent to the at least one biomarker, at least one measurable physical or chemical property of either the at least one biomarker, the detection agent or both will be altered such that the said alteration can be measured by a detector, preferably, comprised in the analyzing unit. However, where analyzing units such as test stripes are used, the detector and the analyzing units may be separate components which are brought together only for the measurement. Based on the detected alteration in the at least one measurable physical or chemical property, the analyzing unit may calculate an intensity value for the at least one biomarker as specified elsewhere herein. Said intensity value can then be transferred for further processing and evaluation to the evaluation unit. Most preferably, the amount of the at least one biomarker can be determined by ELISA, EIA, or RIA based techniques using a detection agent as specified elsewhere herein. Alternatively, an analyzing unit as referred to herein, preferably, comprises means for separating biomarkers, such as chromatographic devices, and means for biomarker determination, such as spectrometry devices. Suitable devices have been described in detail above. Preferred means for compound separation to be used in the system of the present invention include chromatographic devices, more preferably devices for liquid chromatography, HPLC, and/or gas chromatography. Preferred devices for compound determination comprise mass spectrometry devices, more preferably, GC-MS, LC-MS, direct infusion mass spectrometry, FT-ICR-MS, CE-MS, HPLC-MS, quadrupole mass spectrometry, sequentially coupled mass spectrometry (including MS-MS or MS-MS-MS), ICP-MS, Py-MS or TOF. The separation and determination means are, preferably, coupled to each other. Most preferably, LC-MS and/or GC-MS is used in the analyzing unit referred to in accordance with the present invention.

The evaluation unit of the device of the present invention, preferably, comprises a data processing device or computer which is adapted to execute rules for carrying out the comparison as specified elsewhere herein. Moreover, the evaluation unit, preferably, comprises a database with stored references. A database as used herein comprises the data collection on a suitable storage medium. Moreover, the database, preferably, further comprises a database management system. The database management system is, preferably, a network-based, hierarchical or object-oriented database management system. Furthermore, the database may be a federal or integrated database. More preferably, the database will be implemented as a distributed (federal) system, e.g. as a Client-Server-System. More preferably, the database is structured as to allow a search algorithm to compare a test data set with the data sets comprised by the data collection. Specifically, by using such an algorithm, the database can be searched for similar or identical data sets being indicative for bone disorder (e.g. a query search). Thus, if an identical or similar data set can be identified in the data collection, the test data set will be associated with bone disorder. The evaluation unit may also preferably comprise or be operatively linked to a further database with recommendations for therapeutic or preventive interventions or life style adaptations based on the established diagnosis of bone disorder. Said further database can be, preferably, automatically searched with the diagnostic result obtained by the evaluation unit in order to identify suitable recommendations for the subject from which the test sample has been obtained in order to treat or prevent bone disorder.

In a preferred embodiment of the device of the present invention, said stored reference is a reference derived from a subject or a group of subjects known to suffer from bone disorder or a subject or group of subjects which has been brought into contact with at least one compound selected from the group consisting of Sodium perchlorate, Sodium fluoride, Calcitonin (Salmon) or Calcium carbonate, and said data processor executes instructions for comparing the amount of the at least one biomarker determined by the analyzing unit to the stored reference, wherein an essentially identical amount of the at least one biomarker in the test sample in comparison to the reference is indicative for the presence of bone disorder or wherein an amount of the at least one biomarker in the test sample which differs in comparison to the reference is indicative for the absence of bone disorder.

In another preferred embodiment of the device of the present invention, said stored reference is a reference derived from a subject or a group of subjects known not to suffer from bone disorder or a subject or group of subjects which has not been brought into contact with at least one compound selected from the group consisting of 1 Sodium perchlorate, Sodium fluoride, Calcitonin (Salmon) or Calcium carbonate, and said data processor executes instructions for comparing the amount of the at least one biomarker determined by the analyzing unit to the stored reference, wherein an amount of the at least one biomarker in the test sample which differs in comparison to the reference is indicative for the presence of bone disorder or wherein an essentially identical amount of the at least one biomarker in the test sample in comparison to the reference is indicative for the absence of bone disorder.

The device, thus, can also be used without special medical knowledge by medicinal or laboratory staff or patients, in particular when an expert system making recommendations is included. The device is also suitable for near-patient applications since the device can be adapted to a portable format.

The term “kit” refers to a collection of the aforementioned components, preferably, provided separately or within a single container. The container also comprises instructions for carrying out the method of the present invention. These instructions may be in the form of a manual or may be provided by a computer program code which is capable of carrying out the comparisons referred to in the methods of the present invention and to establish a diagnosis accordingly when implemented on a computer or a data processing device. The computer program code may be provided on a data storage medium or device such as an optical or magnetic storage medium (e.g., a Compact Disc (CD), CD-ROM, a hard disk, optical storage media, or a diskette) or directly on a computer or data processing device. A “standard” as referred to in connection with the kit of the invention is an amount of the at least one biomarker when present in solution or dissolved in a predefined volume of a solution resembles the amount of the at least one biomarker which is present (i) in a subject or a group of subjects known to suffer from bone disorder or a subject or group of subjects which has been brought into contact with at least one compound selected from the group consisting of Sodium perchlorate, Sodium fluoride, Calcitonin (Salmon) or Calcium carbonate or (ii) derived from a subject or a group of subjects known to not suffer from therefrom or a subject or group of subjects which has not been brought into contact with at least one compound selected from the group consisting of Sodium perchlorate, Sodium fluoride, Calcitonin (Salmon) or Calcium carbonate.

Advantageously, it has been found in the study underlying the present invention that the amount of at least one biomarker as specified herein allows for diagnosing bone disorder, specifically bone disorder induced by Sodium perchlorate, Sodium fluoride, Calcitonin (Salmon) or Calcium carbonate. The specificity and accuracy of the method will be even more improved by determining an increasing number or even all of the aforementioned biomarkers. A change in the quantitative and/or qualitative composition of the metabolome with respect to these specific biomarkers is indicative for bone disorder even before other signs of the said toxicity are clinically apparent. The morphological, physiological as well as biochemical parameters which are currently used for diagnosing bone disorder are less specific and less sensitive in comparison to the biomarker determination provided by the present invention. Thanks to the present invention, bone disorder of a compound can be more efficiently and reliably assessed. Moreover, based on the aforementioned findings, screening assays for drugs which are useful for the therapy of bone disorder are feasible. In general, the present invention contemplates the use of at least one biomarker in a sample of a subject selected from any one of the Tables 1a, 1b, 1c, 1d, 2a, 2b, 2c, or 2d or a detection agent for said biomarker for diagnosing bone disorder, for determining whether a compound is capable of inducing bone disorder or for identifying a substance capable of treating bone disorder. Further, the present invention, in general, contemplates the use of the at least one biomarker in a sample of a subject or a detection agent therefor for identifying a subject being susceptible for a treatment of bone disorder. Preferred detection agents to be used in this context of the invention are those referred to elsewhere herein. Moreover, the methods of the present invention can be, advantageously, implemented into a device. Furthermore, a kit can be provided which allows for carrying out the methods.

The present invention also relates to a data collection comprising characteristic values for the biomarkers recited in any one of Tables 1a, 1b, 1c, 1d, 2a, 2b, 2c, or 2d. The term “data collection” refers to a collection of data which may be physically and/or logically grouped together. Accordingly, the data collection may be implemented in a single data storage medium or in physically separated data storage media being operatively linked to each other. Preferably, the data collection is implemented by means of a database. Thus, a database as used herein comprises the data collection on a suitable storage medium. Moreover, the database, preferably, further comprises a database management system. The database management system is, preferably, a network-based, hierarchical or object-oriented database management system. Furthermore, the database may be a federal or integrated database. More preferably, the database will be implemented as a distributed (federal) system, e.g. as a Client-Server-System. More preferably, the database is structured as to allow a search algorithm to compare a test data set with the data sets comprised by the data collection. Specifically, by using such an algorithm, the database can be searched for similar or identical data sets being indicative for bone disorder (e.g. a query search). Thus, if an identical or similar data set can be identified in the data collection, the test data set will be associated with bone disorder. Consequently, the information obtained from the data collection can be used to diagnose bone disorder based on a test data set obtained from a subject.

Moreover, the present invention pertains to a data storage medium comprising the said data collection. The term “data storage medium” as used herein encompasses data storage media which are based on single physical entities such as a CD, a CD-ROM, a hard disk, optical storage media, or a diskette. Moreover, the term further includes data storage media consisting of physically separated entities which are operatively linked to each other in a manner as to provide the aforementioned data collection, preferably, in a suitable way for a query search.

The present invention also relates to a system comprising

(a) means for comparing characteristic values of at least one biomarker of a sample operatively linked to

(b) the data storage medium of the present invention.

The term “system” as used herein relates to different means which are operatively linked to each other. Said means may be implemented in a single device or may be implemented in physically separated devices which are operatively linked to each other. The means for comparing characteristic values of the biomarker operate, preferably, based on an algorithm for comparison as mentioned before. The data storage medium, preferably, comprises the aforementioned data collection or database, wherein each of the stored data sets being indicative for bone disorder. Thus, the system of the present invention allows identifying whether a test data set is comprised by the data collection stored in the data storage medium. Consequently, the system of the present invention may be applied as a diagnostic means in diagnosing bone disorder. In a preferred embodiment of the system, means for determining characteristic values of biomakers of a sample are comprised. The term “means for determining characteristic values of biomarkers” preferably relates to the aforementioned devices for the determination of biomarkers such as mass spectrometry devices, ELISA devices, NMR devices or devices for carrying out chemical or biological assays for the analytes.

All references referred to above are herewith incorporated by reference with respect to their entire disclosure content as well as their specific disclosure content explicitly referred to in the above description.

The following Examples are merely for the purposes of illustrating the present invention. They shall not be construed, whatsoever, to limit the scope of the invention in any respect.

### EXAMPLES

### Example

### Biomarkers Associated with Bone Disorder

A group of each 5 male and female rats was dosed once daily with the indicated compounds (see Table 10, below for compounds, applied doses and administeration details) over 28 days.

Each dose group in the studies consisted of five rats per sex. Additional groups of each 5 male and female animals served as controls. Before starting the treatment period, animals, which were 62-64 days old when supplied, were acclimatized to the housing and environmental conditions for 7 days. All animals of the animal population were kept under the same constant temperature (20-24±3° C.) and the same constant humidity (30-70%). The animals of the animal population were fed ad libitum. The food to be used was essentially free of chemical or microbial contaminants. Drinking water was also offered ad libitum. Accordingly, the water was free of chemical and microbial contaminants as laid down in the European Drinking Water Directive 98/83/EG. The illumination period was 12 hours light followed by 12 hours darkness (12 hours light, from 6:00 to 18:00, and 12 hours darkness, from 18:00 to 6:00). The studies were performed in an AAALAC-approved laboratory in accordance with the German Animal Welfare Act and the European Council Directive 86/609/EE. The test system was arranged according to the OECD 407 guideline for the testing of chemicals for repeated dose 28-day oral toxicity study in rodents. The test substances (compounds) in the Tables 1 to 9 below were dosed and administered as described in the Table 10 above.

In the morning of day 7, 14, and 28, blood was taken from the retroorbital venous plexus from fasted anaesthetized animals. From each animal, 1 ml of blood was collected with EDTA as anticoagulant. The samples were centrifuged for generation of plasma. All plasma samples were covered with a N₂ atmosphere and then stored at −80° C. until analysis.

For mass spectrometry-based metabolite profiling analyses plasma samples were extracted and a polar and a non-polar (lipid) fraction was obtained. For GC-MS analysis, the non-polar fraction was treated with methanol under acidic conditions to yield the fatty acid methyl esters. Both fractions were further derivatised with O-methyl-hydroxyamine hydrochloride and pyridine to convert Oxo-groups to O-methyloximes and subsequently with a silylating agent before analysis. In LC-MS analysis, both fractions were reconstituted in appropriate solvent mixtures. HPLC was performed by gradient elution on reversed phase separation columns. Mass spectrometric detection which allows target and high sensitivity MRM (Multiple Reaction Monitoring) profiling in parallel to a full screen analysis was applied as described in WO2003073464.

Steroids and their metabolites were measured by online SPE-LC-MS (Solid phase extraction-LC-MS). Catecholamines and their metabolites were measured by online SPE-LC-MS as described by Yamada et al. (Yamada 2002, Journal of Analytical Toxicology, 26(1): 17-22))

Following comprehensive analytical validation steps, the data for each analyte were normalized against data from pool samples. These samples were run in parallel through the whole process to account for process variability. The significance of treatment group values specific for sex, treatment duration and metabolite was determined by comparing means of the treated groups to the means of the respective untreated control groups using WELCH-test and quantified with treatment ratios versus control and p-values.

The indentification of the most important biomarkers per toxicity pattern was done by a ranking of the analytes in the tables below. Therefore the metabolic changes in reference treatments of a given pattern (shown in the table) were compared with changes of the same metabolite in other unrelated treatments. For each metabolite T-values were obtained for the reference and control treatment and compared by the Welch test to asses whether these two groups are significantly different. The maximum absolute value of the respective TVALUE was taken to indicate the most important metabolite for the pattern.

The changes of the group of plasma metabolites being indicative for bone disorders after treatment of the rats are shown in the following tables:

TABLE 1a
Markers for impaired Bone mineralisation in female rats;
Significant up-regulation changes (p-Value ≦0.2) are marked (*).
For some metabolites (marked with #), additional information
are provided in table 3. All compounds were administered in high dose.

| | Calcitonin | |
|---|---|---|
| | (Salmon) | Calcium carbonate |
| Metabolite | f28 | f28 |
| myo-Inositol-2-phosphate | 1.27* | 1.21* |
| Eicosaenoic acid (C20:1) No 02# | 3.35* | 1.3* |
| Phosphatidylcholine | 1.05* | 1.02* |
| (C18:0, C20:4)# | | |
| Coenzyme Q10 | 1.03* | 1.02* |
| Sphingomyelin (d18:2, C16:0)# | 1.11* | 1.11* |
| 3-Hydroxybutyrate | 1.32* | 1.09* |
| Urea | 1.36* | 1.15* |
| Phosphate (inorganic and organic | 1.31* | 1.28* |
| phosphates) | | |

TABLE 1b
Markers for impaired Bone mineralisation in female rats;
Significant down-regulation changes (p-Value ≦0.2) are marked (*).
For some metabolites (marked with #), additional information are
provided in table 3. All compounds were administered in high dose.

| | Calcitonin | |
|---|---|---|
| | (Salmon) | Calcium carbonate |
| Metabolite | f28 | f28 |
| Phenylalanine | 0.88* | 0.93* |
| Lysophosphatidylcholine | 0.91* | 0.82* |
| (C17:0)# | | |
| Glucose | 0.95* | 0.93* |
| Threonic acid | 0.75* | 0.86* |
| 4-Hydroxyphenylpyruvate | 0.63* | 0.23* |
| Methionine | 0.85* | 0.91* |
| Creatine | 0.92* | 0.48* |
| Alanine | 0.75* | 0.79* |
| 18-Hydroxy-11- | 0.85* | 0.69* |
| deoxycorticosterone | | |
| Creatinine | 0.58* | 0.43* |

TABLE 1c
Markers for impaired Bone mineralisation in male rats;
Significant up-regulation changes (p-Value ≦0.2) are marked (*).
For some metabolites (marked with #), additional information are
provided in table 3. All compounds were administered in high dose.

| | Calcitonin | |
|---|---|---|
| | (Salmon) | Calcium carbonate |
| Metabolite | m28 | m28 |
| Phosphatidylcholine | 1.24* | 1.06* |
| (C16:0, C16:0)# | | |
| Phosphate (inorganic and organic | 1.35* | 1.17* |
| phosphates) | | |
| myo-Inositol-2-phosphate | 1.41* | 1.16* |
| Glycine | 0.86 | 1.31* |

TABLE 1d
Markers for impaired Bone mineralisation in male rats;
Significant down-regulation changes (p-Value ≦0.2) are marked (*).
For some metabolites (marked with #), additional information are
provided in table 3. All compounds were administered in high dose.

| | Calcitonin | |
|---|---|---|
| | (Salmon) | Calcium carbonate |
| Metabolite | m28 | m28 |
| TAG No 07# | 0.78* | 0.71* |
| Pyruvate | 0.76* | 0.77* |
| Heptadecanoic acid (C17:0) | 0.77* | 0.76* |
| TAG (C18:1, C18:2)# | 0.7* | 0.75* |
| Hippuric acid | 0.48* | 0.71* |
| 18-Hydroxy-11- | 0.24* | 0.61* |
| deoxycorticosterone | | |
| 5-Oxoproline | 0.94* | 0.87* |
| TAG No 01# | 0.66* | 0.55* |
| Linoleic acid | 0.82* | 0.67* |
| (C18:cis[9, 12]2) | | |
| TAG (C18:2, C18:3)# | 0.78* | 0.64* |
| Malate | 0.8* | 0.54* |
| Lactate | 0.7* | 0.95* |
| Alanine | 0.78* | 0.79* |
| 16-Methylheptadecanoic | 0.59* | 0.66* |
| acid | | |
| Coenzyme Q9 | 0.83* | 0.72* |
| Glycerol, lipid fraction | 0.86* | 0.66* |
| TAG (C18:2, C18:2)# | 0.6* | 0.74* |
| Linolenic acid | 0.61* | 0.81* |
| (C18:cis[9, 12, 15]3) | | |
| 17-Methyloctadecanoic acid | 0.65* | 0.92* |
| TAG No 05# | 0.68* | 0.71* |
| gamma-Linolenic acid | 0.98 | 0.7* |
| (C18:cis[6, 9, 12]3) | | |
| DAG (C18:1, C18:2)# | 0.63* | 0.87* |
| Coenzyme Q10 | 0.74* | 0.84* |
| Glycine | 0.86* | 1.31 |
| TAG (DAG-Fragment)# | 0.7* | 0.72* |
| TAG No 059# | 0.65* | 0.68* |
| Methionine | 0.88* | 0.81* |

TABLE 2a
Markers for Bone osteoblast inhibitors in male rats; Significant
up-regulation changes (p-Value ≦ 0.2) are marked (*). For
some metabolites (marked with #), additional information are
provided in table 3. All compounds were administered in high dose.

| | Sodium perchlorate | | | Sodium fluoride | | |
|---|---|---|---|---|---|---|
| Metabolite | m7 | m14 | m28 | m7 | m14 | m28 |
| Isoleucine | 0.97 | 1.16 * | 1.37 * | 1.2 * | 1.16 * | 1.27 * |
| Leucine | 1.16 * | 1.36 * | 1.22 * | 1.19 * | 1.19 | 1.25 * |
| Dodecanol | 1.05 * | 1.09 | 0.96 | 1.24 * | 1.53 * | 1.49 * |
| Phosphate | 0.95 | 1.13 * | 1.31 | 1.25 * | 1.29 * | 1.26 * |
| (inorganic | | | | | | |
| and organic | | | | | | |
| phosphates) | | | | | | |

TABLE 2b
Markers for Bone osteoblast inhibitors in male rats; Significant
down-regulation changes (p-Value ≦ 0.2) are marked (*). For
some metabolites (marked with #), additional information are
provided in table 3. All compounds were administered in high dose.

| | Sodium perchlorate | | | Sodium fluoride | | |
|---|---|---|---|---|---|---|
| Metabolite | m7 | m14 | m28 | m7 | m14 | m28 |
| Ketoleucine | 0.76 * | 0.74 * | 0.78 * | 0.87 * | 0.71 * | 0.97 |
| 18-Hydroxy- | 0.88 | 0.21 * | 0.44 * | 0.73 | 0.41 * | 0.39 * |
| 11-deoxy- | | | | | | |
| corticosterone | | | | | | |

TABLE 2c
Markers for Bone ostoblast inhibitors in male rats; Significant
up-regulation changes (p-Value ≦0.2) are marked (*). For some
metabolites (marked with #), additional information are provided
in table 3. All compounds were administered in high dose.

| | | Sodium |
|---|---|---|
| | Sodium perchlorate | fluoride |
| Metabolite | m28 | m28 |
| TAG (C16:0, C18:1, C18:3)# | 5.73* | 1.58* |
| Cholesterolester No 01# | 1.13* | 1.07* |
| Threonine | 1.1* | 1.18* |
| TAG (C16:0, C18:2)# | 2.04* | 1.21* |
| TAG (C18:2, C18:2)# | 3.09* | 1.54* |
| alpha-Tocopherol | 1.49* | 1.45* |
| TAG (C18:1, C18:2)# | 2.33* | 1.74* |
| Isoleucine | 1.37* | 1.27* |
| Phosphate (inorganic and organic | 1.31 | 1.26* |
| phosphates) | | |
| Ornithine | 1.33* | 1.29* |
| Oleic acid (C18:cis[9]1) | 1.02 | 1.75* |
| 14-Methylhexadecanoic acid | 0.86 | 1.27* |
| TAG (DAG-Fragment)# | 2.84* | 2.08* |
| TAG No 059# | 3.68* | 2.7* |
| Phenylalanine | 1.13 | 1.13* |

TABLE 2d
Markers for Bone ostoblast inhibitors in male rats; Significant
down-regulation changes (p-Value ≦0.2) are marked (*). For
some metabolites (marked with #), additional information are
provided in table 3. All compounds were administered in high dose.

| | Sodium | Sodium |
|---|---|---|
| | perchlorate | fluoride |
| Metabolite | m28 | m28 |
| 18-Hydroxy-11-deoxycorticosterone | 0.44* | 0.39* |
| Glucose | 0.78 | 0.75* |
| Glycerol, polar fraction | 1 | 0.83* |

TABLE 3
Chemical/physical properties of selected analytes. These biomarkers are characterized herein by chemical and physical properties.

| Metabolite | Fragmentation pattern (GC-MS) and description |
|---|---|
| 3-O-Methylsphingosine (d18:1) | 3-O-Methylsphingosine (d18:1) exhibits the following characteristic ionic fragments when detected |
| | with GC/MS, applying electron impact (EI) ionization mass spectrometry, after acidic |
| | methanolysis and derivatisation with 2% O-methylhydroxylamine-hydrochlorid in pyridine and |
| | subsequently with N-methyl-N-trimethylsilyltrifluoracetamid: MS (EI, 70 eV): m/z (%): 204 (100), |
| | 73 (18), 205 (16), 206 (7), 354 (4), 442 (1). |
| 5-O-Methylsphingosine (d18:1) | 5-O-Methylsphingosine (d18:1) exhibits the following characteristic ionic fragments when detected |
| | with GC/MS, applying electron impact (EI) ionization mass spectrometry, after acidic |
| | methanolysis and derivatisation with 2% O-methylhydroxylamine-hydrochlorid in pyridine and |
| | subsequently with N-methyl-N-trimethylsilyltrifluoracetamid: MS (EI, 70 eV): m/z (%): 250 (100), |
| | 73 (34), 251 (19), 354 (14), 355 (4), 442 (1). |
| Cholesterolester No 01 | Metabolite belongs to the class of cholesterolesters. It exhibits the following characteristic ionic |
| | species when detected with LC/MS, applying electro-spray ionization (ESI) mass spectrometry: |
| | mass-to-charge ratio (m/z) of the positively charged ionic species is 369.2 (+/−0.5). |
| Choline plasmalogen No 01 | Metabolite belongs to the class of choline plasmalogens. It exhibits the following characteristic |
| | ionic species when detected with LC/MS, applying electro-spray ionization (ESI) mass spectrometry: |
| | mass-to-charge ratio (m/z) of the positively charged ionic species is 772.6 (+/−0.5). |
| Choline plasmalogen No 02 | Metabolite belongs to the class of choline plasmalogens. It exhibits the following characteristic |
| | ionic species when detected with LC/MS, applying electro-spray ionization (ESI) mass spectrometry: |
| | mass-to-charge ratio (m/z) of the positively charged ionic species is 767 (+/−0.5). |
| Choline plasmalogen No 03 | Metabolite belongs to the class of choline plasmalogens. It exhibits the following characteristic |
| | ionic species when detected with LC/MS, applying electro-spray ionization (ESI) mass spectrometry: |
| | mass-to-charge ratio (m/z) of the positively charged ionic species is 768.8 (+/−0.5). |
| DAG (C18:1,C18:2) | DAG (C18:1,C18:2) represents the sum parameter of diacylglycerols containing the combination |
| | of a C18:1 fatty acid unit and a C18:2 fatty acid unit. The mass-to-charge ratio (m/z) of the |
| | ionised species is 641.6 Da (+/−0.5 Da). |
| Eicosaenoic acid (C20:1) No | Eicosaenoic acid (C20:1) exhibits the following characteristic ionic fragments when detected |
| 02 | with GC/MS, applying electron impact (EI) ionization mass spectrometry, after acidic meth- |
| | anolysis and derivatisation with 2% O-methylhydroxylamine-hydrochlorid in pyridine and sub- |
| | sequently with N-methyl-N-trimethylsilyltrifluoracetamid: MS (EI, 70 eV): m/z (%): 55 (100), 69 |
| | (75), 41 (57), 83 (54), 74 (53), 97 (45), 110 (20), 292 (13), 293 (13), 124 (12), 250 (9), 152 (8), |
| | 138 (8), 208 (7), 324 (2). |
| Glycerol phosphate, lipid fraction | Glycerol phosphate, lipid fraction represents the sum parameter of metabolites containing a |
| | glycerol-2-phosphate or a glycerol-3-phosphate moiety and being present in the lipid fraction |
| | after extraction and separation of the extract into a polar and a lipid fraction. |
| Lysophosphatidylcholine (C17:0) | Lysophosphatidylcholine (C17:0) represents the sum parameter of lysoglycerophosphorylcholines |
| | containing a C17:0 fatty acid unit. If detected with LC/MS, applying electro-spray ionization |
| | (ESI) mass spectrometry, the mass-to-charge ratio (m/z) of the positively charged ionic species |
| | is 510.4 Da (+/−0.5 Da). |
| Lysophosphatidylcholine | Lysophosphatidylcholine (C18:0) represents the sum pa-rameter of lysoglycerophosphorylcholines |
| (C18:0) | containing a C18:0 fatty acid unit. If detected with LC/MS, applying electro-spray ionization |
| | (ESI) mass spectrometry, the mass-to-charge ratio (m/z) of the positively charged ionic species |
| | is 546.6 Da (+/−0.5 Da). |
| Lysophosphatidylcholine | Lysophosphatidylcholine (C18:1) represents the sum pa-rameter of lysoglycerophosphorylcholines |
| (C18:1) | containing a C18:1 fatty acid unit. If detected with LC/MS, applying electro-spray ionization |
| | (ESI) mass spectrometry, the mass-to-charge ratio (m/z) of the positively charged ionic species |
| | is 522.2 Da (+/−0.5 Da). |
| Lysophosphatidylcholine | Lysophosphatidylcholine (C18:2) represents the sum parameter of lysoglycerophosphorylcholines |
| (C18:2) | containing a C18:2 fatty acid unit. If detected with LC/MS, applying electro- |
| | spray ionization (ESI) mass spectrometry, the mass-to-charge ratio (m/z) of the positively |
| | charged ionic species is 542.4 Da (+/−0.5 Da). |
| Lysophosphatidylcholine | Lysophosphatidylcholine (C20:4) represents the sum pa-rameter of lysoglycerophosphorylcholines |
| (C20:4) | containing a C20:4 fatty acid unit. If detected with LC/MS, applying electro-spray ionization |
| | (ESI) mass spectrometry, the mass-to-charge ratio (m/z) of the positively charged ionic species |
| | is 544.4 Da (+/−0.5 Da). |
| Lysophosphatidylethanolamine (C22:5) | Lysophosphatidylethanolamine (C22:5) exhibits the following characteristic ionic species when |
| | detected with LC/MS, applying electro-spray ionization (ESI) mass spectrometry: mass-to- |
| | charge ratio (m/z) of the positively charged ionic species is 528.2 (+/−0.5). |
| Phosphatidylcholine | Phosphatidylcholine (C16:0/C16:0) represents the sum parameter of glycerophosphorylcholines |
| (C16:0,C16:0) | containing either the combination of of two C16:0 fatty acid units. The mass-to-charge ratio |
| | (m/z) of the ionised species is 734.8 Da (+/−0.5 Da). |
| Phosphatidylcholine | Phosphatidylcholine (C16:0,C20:5) exhibits the following characteristic ionic species when detected |
| (C16:0,C20:5) | with LC/MS, applying electro-spray ionization (ESI) mass spectrometry: mass-to-charge |
| | ratio (m/z) of the positively charged ionic species is 780.8 (+/−0.5). |
| Phosphatidylcholine | Phosphatidylcholine (C16:1,C18:2) represents the sum parameter of glycerophosphorylcholines |
| (C16:1,C18:2) | containing the combination of a C16:1 fatty acid unit and a C18:2 fatty |
| | acid unit. If detected with LC/MS, applying electro-spray ionization (ESI) mass spectrometry, |
| | the mass-to-charge ratio (m/z) of the positively charged ionic species is 756.8 Da (+/−0.5 Da). |
| Phosphatidylcholine | Phosphatidylcholine (C18:0,C18:1) represents the sum parameter of glycerophosphorylcholines |
| (C18:0,C18:1) | containing the combination of a C18:0 fatty acid unit and a C18:1 fatty acid unit. If detected |
| | with LC/MS, applying electro-spray ionization (ESI) mass spectrometry, the mass-to-charge |
| | ratio (m/z) of the positively charged ionic species is 788.6 Da (+/−0.5 Da). |
| Phosphatidylcholine | Phosphatidylcholine (C18:0,C18:2) represents the sum parameter of glycerophosphorylcholines |
| (C18:0,C18:2) | containing the combination of a C18:0 fatty acid unit and a C18:2 fatty acid unit. If detected |
| | with LC/MS, applying electro-spray ionization (ESI) mass spectrometry, the mass-to-charge |
| | ratio (m/z) of the positively charged ionic species is 786.6 Da (+/−0.5 Da). |
| Phosphatidylcholine | Phosphatidylcholine (C18:0,C20:3) exhibits the following characteristic ionic species when detected |
| (C18:0,C20:3) | with LC/MS, applying electro-spray ionization (ESI) mass spectrometry: mass-to-charge |
| | ratio (m/z) of the positively charged ionic species is 812.6 (+/−0.5). |
| Phosphatidylcholine | Phosphatidylcholine (C18:0,C20:4) represents the sum parameter of glycerophosphorylcholines |
| (C18:0,C20:4) | containing the combination of a C18:0 fatty acid unit and a C20:4 fatty acid unit. If detected |
| | with LC/MS, applying electro-spray ionization (ESI) mass spectrometry, the mass-to-charge |
| | ratio (m/z) of the positively charged ionic species is 810.8 Da (+/−0.5 Da). |
| Phosphatidylcholine | Phosphatidylcholine (C18:0,C22:6) represents the sum parameter of glycerophosphorylcholines |
| (C18:0,C22:6) | containing the combination of a C18:0 fatty acid unit and a C22:6 fatty acid unit. If detected |
| | with LC/MS, applying electro-spray ionization (ESI) mass spectrometry, the mass-to-charge |
| | ratio (m/z) of the positively charged ionic species is 834.8 Da (+/−0.5 Da). |
| Phosphatidylcholine | Phosphatidylcholine (C16:0/C20:3 C18:1/C18:2) represents the sum parameter of glycerophosphorylcholines |
| (C18:1,C18:2) | containing the combination of a C18:1 fatty acid unit and a C18:2 fatty |
| | acid unit. The mass-to-charge ratio (m/z) of the ionised species is 784.6 Da (+/−0.5 Da). |
| Phosphatidylcholine | Phosphatidylcholine (C16:0/C22:6 C18:2/C20:4) represents the sum parameter of glycerophosphorylcholines |
| (C18:2,C20:4) | containing either the combination of a C16:0 fatty acid unit and a C22:6 |
| | fatty acid unit or the combination of a C18:2 fatty acid unit and a C20:4 fatty acid unit. The |
| | mass-to-charge ratio (m/z) of the ionised species is 806.6 Da (+/−0.5 Da). |
| Phosphatidylcholine No 02 | Metabolite belongs to the class of glycerophosphocholines. It exhibits the following characteristic |
| | ionic species when detected with LC/MS, applying electro-spray ionization (ESI) mass spectrometry: |
| | mass-to-charge ratio (m/z) of the positively charged ionic species is 808.4 (+/−0.5). |
| Phosphatidylcholine No 04 | Metabolite belongs to the class of glycerophosphocholines. It exhibits the following characteristic |
| | ionic species when detected with LC/MS, applying electro-spray ionization (ESI) mass spectrometry: |
| | mass-to-charge ratio (m/z) of the positively charged ionic species is 796.8 (+/−0.5). |
| Sphingomyelin (d18:1,C23:0) | Sphingomyelin (d18:1,C23:0) exhibits the following characteristic ionic species when detected |
| | with LC/MS, applying electro-spray ionization (ESI) mass spectrometry: mass-to-charge ratio |
| | (m/z) of the positively charged ionic species is 801.8 (+/−0.5). |
| Sphingomyelin (d18:1,C24:0) | Sphingomyelin (d18:1,C24:0) represents the sum pa-rameter of sphingomyelins containing the |
| | combination of a d18:1 long-chain base unit and a C24:0 fatty acid unit. If detected with LC/MS, |
| | applying electro-spray ionization (ESI) mass spectrometry, the mass-to-charge ratio (m/z) of the |
| | positively charged ionic species is 815.8 Da (+/−0.5 Da). |
| Sphingomyelin (d18:2,C16:0) | Sphingomyelin (d18:2,C16:0) exhibits the following characteristic ionic species when detected |
| | with LC/MS, applying electro-spray ionization (ESI) mass spectrometry: mass-to-charge ratio |
| | (m/z) of the positively charged ionic species is 723.6 (+/−0.5). |
| Sphingomyelin (d18:2,C18:0) | Sphingomyelin (d18:2,C18:0) exhibits the following characteristic ionic species when detected |
| | with LC/MS, applying electro-spray ionization (ESI) mass spectrometry: mass-to-charge ratio |
| | (m/z) of the positively charged ionic species is 729.8 (+/−0.5). |
| TAG (C16:0,C16:1) | Metabolite represents the sum of triacylglycerides containing the combination of a C16:0 fatty |
| | acid unit and a C16:1 fatty acid unit. It exhibits the following characteristic ionic species when |
| | detected with LC/MS, applying electro-spray ionization (ESI) mass spectrometry: mass-to- |
| | charge ratio (m/z) of the positively charged ionic species is 549.6 (+/−0.5). |
| TAG (C16:0,C18:1,C18:3) | TAG (C16:0,C18:1,C18:3) exhibits the following characteristic ionic species when detected with |
| | LC/MS, applying electro-spray ionization (ESI) mass spectrometry: mass-to-charge ratio (m/z) |
| | of the positively charged ionic species is 855.6 (+/−0.5). |
| TAG (C16:0,C18:2) | Metabolite represents the sum of triacylglycerides containing the combination of a C16:0 fatty |
| | acid unit and a C18:2 fatty acid unit. It exhibits the following characteristic ionic species when |
| | detected with LC/MS, applying electro-spray ionization (ESI) mass spectrometry: mass-to- |
| | charge ratio (m/z) of the positively charged ionic species is 575.6 (+/−0.5). |
| TAG (C18:1,C18:2) | Metabolite represents the sum of triacylglycerides containing the combination of a C18:1 fatty |
| | acid unit and a C18:2 fatty acid unit. It exhibits the following characteristic ionic species when |
| | detected with LC/MS, applying electro-spray ionization (ESI) mass spectrometry: mass-to- |
| | charge ratio (m/z) of the positively charged ionic species is 601.6 (+/−0.5). |
| TAG (C18:2,C18:2) | Metabolite represents the sum of triacylglycerides containing the combination of a C18:2 fatty |
| | acid unit and a C18:2 fatty acid unit. It exhibits the following characteristic ionic species when |
| | detected with LC/MS, applying electro-spray ionization (ESI) mass spectrometry: mass-to- |
| | charge ratio (m/z) of the positively charged ionic species is 599.6 (+/−0.5). |
| TAG (C18:2,C18:3) | Metabolite represents the sum of triacylglycerides containing the combination of a C18:2 fatty |
| | acid unit and a C18:3 fatty acid unit. It exhibits the following characteristic ionic species when |
| | detected with LC/MS, applying electro-spray ionization (ESI) mass spectrometry: mass-to- |
| | charge ratio (m/z) of the positively charged ionic species is 597.6 (+/−0.5). |
| TAG (DAG-Fragment) | Metabolite belongs to the class of triacylglycerides. It exhibits the following characteristic ionic |
| | species when detected with LC/MS, applying electro-spray ionization (ESI) mass spectrometry: |
| | mass-to-charge ratio (m/z) of the positively charged ionic species is 600.6 (+/−0.5). |
| TAG No 01 | Metabolite belongs to the class of triacylglycerides. It exhibits the following characteristic ionic |
| | species when detected with LC/MS, applying electro-spray ionization (ESI) mass spectrometry: |
| | mass-to-charge ratio (m/z) of the positively charged ionic species is 547.6 (+/−0.5). |
| TAG No 02 | Metabolite belongs to the class of triacylglycerides. It exhibits the following characteristic ionic |
| | species when detected with LC/MS, applying electro-spray ionization (ESI) mass spectrometry: |
| | mass-to-charge ratio (m/z) of the positively charged ionic species is 695.6 (+/−0.5). |
| TAG No 05 | Metabolite belongs to the class of triacylglycerides. It exhibits the following characteristic ionic |
| | species when detected with LC/MS, applying electro-spray ionization (ESI) mass spectrometry: |
| | mass-to-charge ratio (m/z) of the positively charged ionic species is 879.6 (+/−0.5). |
| TAG No 059 | Metabolite belongs to the class of triacylglycerides. It exhibits the following characteristic ionic |
| | species when detected with LC/MS, applying electro-spray ionization (ESI) mass spectrometry: |
| | mass-to-charge ratio (m/z) of the positively charged ionic species is 904 (+/−0.5). |
| TAG No 07 | Metabolite belongs to the class of triacylglycerides. It exhibits the following characteristic ionic |
| | species when detected with LC/MS, applying electro-spray ionization (ESI) mass spectrometry: |
| | mass-to-charge ratio (m/z) of the positively charged ionic species is 853.6 (+/−0.5). |

TABLE 4
Compounds and dosage regimens

| Compound | Dose | Synonym | CAS no | Dosage administered | Details |
|---|---|---|---|---|---|
| Calcitonin | high | na | 47931- | 8 μg/kg body | in aqua bidest. containing |
| (Salmon) | | | 85-1 | weight subcutaneous | 0.5% CMC (Tylose CB30000), |
| | | | | | administration volume: 10 ml/kg |
| | | | | | body weight |
| Calcium | high | na | 471-34-1 | 45000 ppm in | mixture in the diet |
| carbonate | | | | the diet | |
| Sodium | high | na | 7601-89-0 | 20 mg/kg | in 0.9% aqueous NaCl- |
| perchlorate | | | | body weight | solution, administration volume: |
| | | | | by gavage | 10 ml/kg body weight |
| Sodium | high | na | 7681-49-4 | 30 mg/kg | in drinking water, administration |
| fluoride | | | | body weight | volume: 10 ml/kg bw |
| | | | | by gavage | |

1. A method for diagnosing bone disorder comprising:
(a) determining the amount of at least one biomarker selected from any one of Tables 1a, 1b, 1c, 1d, 2a, 2b, 2c, or 2d in a test sample of a subject suspected to suffer from bone disorder, and
(b) comparing the amounts determined in step (a) to a reference, whereby bone disorder is to be diagnosed.

2. The method of claim 1, wherein said subject has been brought into contact with a compound suspected to be capable of inducing bone disorder.

3. A method of determining whether a compound is capable of inducing bone disorder in a subject comprising:
(a) determining in a sample of a subject which has been brought into contact with a compound suspected to be capable of inducing bone disorder the amount of at least one biomarker selected from any one of Tables 1a, 1b, 1c, 1d, 2a, 2b, 2c, or 2d hematopoietic and
(b) comparing the amounts determined in step (a) to a reference, whereby the capability of the compound to induce bone disorder is determined.

4. The method of claim 2, wherein said compound is at least one compound selected from the group consisting of: Sodium perchlorate, Sodium fluoride, Calcitonin (Salmon) and Calcium carbonate.

5. The method of claim 1, wherein said reference is derived from (i) a subject or group of subjects which suffers from bone disorder or (ii) a subject or group of subjects which has been brought into contact with at least one compound selected from the group consisting of: Sodium perchlorate, Sodium fluoride, Calcitonin (Salmon) and Calcium carbonate.

6. The method of claim 5, wherein essentially identical amounts for the biomarkers in the test sample and the reference are indicative for bone disorder.

7. The method of claim 1, wherein said reference is derived from (i) a subject or group of subjects known to not suffer from bone disorder or (ii) a subject or group of subjects which has not been brought into contact with at least one compound selected from the group consisting of: Sodium perchlorate, Sodium fluoride, Calcitonin (Salmon) and Calcium carbonate.

8. The method of claim 1, wherein said reference is a calculated reference for the biomarkers for a population of subjects.

9. The method of claim 7, wherein amounts for the biomarkers which differ in the test sample in comparison to the reference are indicative for bone disorder.

10. A method of identifying a substance for treating bone disorder comprising the steps of:
(a) determining in a sample of a subject suffering from bone disorder which has been brought into contact with a candidate substance suspected to be capable of treating bone disorder the amount of at least one biomarker selected from any one of Tables 1a, 1b, 1c, 1d, 2a, 2b, 2c, or 2d; and
(b) comparing the amounts determined in step (a) to a reference, whereby a substance capable of treating bone disorder is to be identified.

11. The method of claim 10, wherein said reference is derived from (i) a subject or group of subjects which suffers from bone disorder or (ii) a subject or group of subjects which has been brought into contact with at least one compound selected from the group consisting of: Sodium perchlorate, Sodium fluoride, Calcitonin (Salmon) and Calcium carbonate.

12. The method of claim 11, wherein amounts for the biomarkers which differ in the test sample and the reference are indicative for a substance capable of treating bone disorder.

13. The method of claim 10, wherein said reference is derived from (i) a subject or group of subjects known to not suffer from bone disorder or (ii) a subject or group of subjects which has not been brought into contact with at least one compound selected from the group consisting of: Sodium perchlorate, Sodium fluoride, Calcitonin (Salmon) and Calcium carbonate.

14. The method of claim 10, wherein said reference is a calculated reference for the biomarkers in a population of subjects.

15. The method of claim 13, wherein essentially identical amounts for the biomarkers in the test sample and the reference are indicative for a substance capable of treating bone disorder.

16. (canceled)

17. A device for diagnosing bone disorder in a sample of a subject suspected to suffer therefrom comprising:
(a) an analyzing unit comprising a detection agent for at least one biomarker selected from any one of Tables 1a, 1b, 1c, 1d, 2a, 2b, 2c, or 2d which allows for determining the amount of the said biomarker present in the sample; and, operatively linked thereto,
(b) an evaluation unit comprising a stored reference and a data processor which allows for comparing the amount of the said at least one biomarker determined by the analyzing unit to the stored reference, whereby bone disorder is diagnosed.

18. The device of claim 17, wherein said stored reference is a reference derived from a subject or a group of subjects known to suffer from bone disorder or a subject or group of subjects which has been brought into contact with at least one compound selected from the group consisting of Sodium perchlorate, Sodium fluoride, Calcitonin (Salmon) and Calcium carbonate and said data processor executes instructions for comparing the amount of the at least one biomarker determined by the analyzing unit to the stored reference, wherein an essentially identical amount of the at least one biomarker in the test sample in comparison to the reference is indicative for the presence of bone disorder or wherein an amount of the at least one biomarker in the test sample which differs in comparison to the reference is indicative for the absence of bone disorder.

19. The device of claim 17, wherein said stored reference is a reference derived from a subject or a group of subjects known to not suffer from bone disorder or a subject or group of subjects which has not been brought into contact with at least one compound selected from the group consisting of Sodium perchlorate, Sodium fluoride, Calcitonin (Salmon) and Calcium carbonate and said data processor executes instructions for comparing the amount of the at least one biomarker determined by the analyzing unit to the stored reference, wherein an amount of the at least one biomarker in the test sample which differs in comparison to the reference is indicative for the presence of bone disorder or wherein an essentially identical amount of the at least one biomarker in the test sample in comparison to the reference is indicative for the absence of bone disorder.

20. A kit for diagnosing bone disorder comprising a detection agent for the at least one biomarker selected from any one of Tables 1a, 1b, 1c, 1d, 2a, 2b, 2c, or 2d and standards for the at least one biomarker the concentration of which is derived from (i) a subject or a group of subjects known to suffer from bone disorder or a subject or group of subjects which has been brought into contact with at least one compound selected from the group consisting of Sodium perchlorate, Sodium fluoride, Calcitonin (Salmon) and Calcium carbonate or derived (ii) from a subject or a group of subjects known to not suffer from bone disorder or a subject or group of subjects which has not been brought into contact with at least one compound selected from the group consisting of Sodium perchlorate, Sodium fluoride, Calcitonin (Salmon) and Calcium carbonate.
